(19) United States
(12) Patent Application Publication
(10) Pub. No.: US 2019/0316169 A1
(43) Pub. Date: Oct. 17, 2019

(54) METHODS, COMPOSITIONS, AND KITS FOR DETECTING A CELL IN A SAMPLE

(71) Applicant: Sonanutech Inc., Davis, CA (US)

(72) Inventors: Sudheendra Lakshmana, Davis, CA (US); Alice Ngo, West Sacramento, CA (US); Laura Nicole Putnam, Davis, CA (US); Alexander Thornton-Dunwoody, Davis, CA (US); Leslie Anne Jones, Davis, CA (US)

(73) Assignee: Sonanutech Inc., Davis, CA (US)

(21) Appl. No.: 16/462,237

(22) PCT Filed: Nov. 24, 2017
(86) PCT No.: PCT/US17/63199

§ 371 Date: May 19, 2019

Related U.S. Application Data
(60) Provisional application No. 62/427,084, filed on Nov. 28, 2016.
(60) Provisional application No. 62/450,361, filed on Jan. 25, 2017.
(60) Provisional application No. 62/453,099, filed on Feb. 1, 2017.
(60) Provisional application No. 62/518,600, filed on Jun. 13, 2017.
(60) Provisional application No. 62/518,598, filed on Jun. 13, 2017.

(51) Int. Cl. C12Q 1/04; C12Q 1/70; C12Q 1/18; C12Q 1/68; G01N 33/04; G01N 33/569

(52) U.S. Cl. CPC C12Q 1/04; C12Q 1/701; C12Q 1/18; C12Q 2600/158; G01N 33/04; G01N 33/569; C12Q 1/68

(57) ABSTRACT

Provided are methods, compositions, devices, and kits for detecting bacteria in a sample. The methods, compositions, devices, and kits are specially useful for diagnosis and prognosis of an individual suspected of having bacterial infection. The methods, compositions, devices, and kits are applicable to a wide variety of areas, for example, medical diagnostics, industries involving bacterial fermentation, dairy and wine industries, prevention of bioterrorism.

### CROSS-REFERENCE TO RELATED APPLICATIONS

This application claims the priority under 35 U.S.C. § 119(e) to U.S. Provisional Patent Application Ser. No. 62/427,084 filed Nov. 26, 2016; U.S. Provisional Patent Application Ser. No. 62/450,361 filed Jan. 25, 2017; U.S. Provisional Patent Application Ser. No. 62/453,099 filed Feb. 1, 2017; U.S. Provisional Patent Application Ser. No. 62/5185,98 filed Jun. 13, 2017; and U.S. Provisional Patent Application Ser. No. 62/518,600 filed Jun. 13, 2017, the entire contents of each of which are incorporated by reference herein.

### STATEMENT REGARDING FEDERALLY SPONSORED RESEARCH AND

DEVELOPMENT

The invention was made with Government support under Grant Nos. 1534756 awarded by the National Science Foundation. The government has certain rights in the invention.

### FIELD OF THE INVENTION

Provided are methods, compositions, devices, and kits for detecting bacteria in a sample.

### BACKGROUND OF THE INVENTION

Pathogenic bacteria pose a global health threat and cause extensive morbidity and mortality each year. Contaminated food and water are major sources for infection by bacterial pathogens. The emergence and spread of drug resistance among bacterial pathogens is a cause for concern and has been observed among various pathogens such as the gram negatives as well as the gram-positive bacteria.

There is a need for rapid and effective diagnostics to contain the spread of bacterial pathogens. Bacterial culture remains one of the most common methods for bacterial detection and drug resistance profiling. However, this leads to a diagnostic time delay for bacteria with a slow growth rate. Furthermore, certain pathogens require specialized biohazard facilities, prohibiting its wide-spread use. Culture is also limited to culturable bacteria, however viable but non-culturable (VBNC) cells could escape detection.

### SUMMARY OF THE INVENTION

Inventors of the present application have surprisingly and unexpectedly found that treating a nucleic acid or proteins with certain compounds prevent another compound to bind to the same nucleic acid or the protein. In some embodiments, the compounds can comprise a detectable label such as a fluorescent dye. Inventors of the present application also surprisingly and unexpectedly found that in certain embodiments the order of treatment of the different compounds are critical to such differential binding of the compounds to the nucleic acid and proteins. Such findings allow differentially labeling a nucleic acid or a protein in the presence of a different nucleic acid or protein that is pre-treated with a compound. Inventors of the present application also surprisingly and unexpectedly found that in certain embodiments, in addition to differential labelling a virus and/or bacteriophage and cell and/or bacteria it is possible to label viruses and/or phages and inhibit its replication inside a cell and/or bacteria.

In one aspect, provided are methods for detecting the presence of one or more types of cell in a sample. The methods include a) providing one or more types of viruses in which each of said one or more types of viruses bind to one or more specific types of receptor of one or more types of cell if present in the sample to form complexes of virus and cell; b) contacting the sample suspected of comprising the cell with said one or more types of viruses in which one or more types of viruses form complexes with one or more types of cell if present in the sample; c) contacting one or more types of viruses with a first set of one or more compounds to generate one or more types of viruses comprising a nucleic acid comprising the first set of one or more compounds; d) contacting the one or more types of cell with a second set of one or more compounds in which the second set of one or more compounds preferentially interacts with one or more components of the one or more types of cell in the presence of said one or more types of viruses; c) detecting the complexes by detecting the second set of one or more compounds or modifications of the second set of one or more compounds by one or more types of cell. The complexes are indicative of the presence of one or more types of cell in the sample.

In some embodiments, upon contacting one or more types of viruses with the first set of one or more compounds inhibits replication of one or more types of viruses in one or more types of cell. In some embodiments, contacting the one or more types of viruses with a first set of one or more compounds is done prior to contacting said sample suspected of comprising said cell with said one or more types of viruses. In some embodiments, one or more types of cell are one or more types of bacteria, and one or more types of viruses are one or more types of bacteriophage that bind to one or more specific types of receptor of the one or more types of bacteria. In some embodiments, the second set of one or more compounds preferentially binds to the nucleic acid of said one or more types of bacteria. In some embodiments, the first set of one or more compounds comprises a first detectable label. In some embodiments, the detecting further comprises detecting said first set of one or more compounds. In some embodiments, the first set of one or more compounds is propidium iodide, ethidium bromide, propidium monoazide, ethidium monoazide, a combination of, and/or derivatives thereof.

In some embodiments, the second set of one or more compounds interact with cellular nucleic acid. In some embodiments, the second set of one or more compounds comprise Calcein derivative. In some embodiments, the second set of one or more compounds comprise a substrate for cellular enzymes. In some embodiments, the second set of one or more compounds comprises an antibody specific for a cellular protein. In some embodiments, the second set of one or more compounds comprise a second detectable label. In some embodiments, second set of one or more compounds is a redox compound. In some embodiments, the second set of one or more compounds is Resazurin, Resorufin, Dihydroresorufin, or a combination thereof. In some embodiments, the modification of the second set of one or more compounds comprise a change in the oxidative state of Resazurin, Resorufin, Dihydroresorufin, or a combination thereof In some embodiments, the detecting modifications of the second set of one or more compounds comprise measuring the change of the oxidative status of Resazurin, Resorufin, Dihydroresorufin or a combination thereof. In some embodiments, the methods include measuring any modifications of the second set of one or more compounds by one or more types of cell over a period of time and determining the kinetic profile of the modification of the second set of one or more compounds. The kinetic profile is indicative of the presence of the one or more types of cell. In some embodiments, the method is carried out between about 20° C. and about 50° C. In some embodiments, the method is carried out at about 25° C., 27° C., 30° C., 33° C., 35° C., 37° C., 40° C., 42° C., or 45° C.

In some embodiments, the methods further include incubating the complexes of the one or more types of bacteria and bacteriophage in a media comprising one or more bactericide or bacteriostatic agent prior to detecting said complexes. In some embodiments, the methods further include separating said complexes of said one or more types of bacteria and bacteriophage from said sample prior to incubating in a media comprising one or more bactericide or bacteriostatic agent. In some embodiments, the one or more types of bacteriophages are immobilized on a solid support.

In some embodiments, the one or more compounds from the first set and one or more compounds from second set are same. In some embodiments, first set of one or more compounds are different from second set of one or more compounds.

In one aspect, provided are methods for detecting the presence of one or more bactericide or bacteriostatic agent resistant bacteria in a sample. The methods include a) providing a first sample comprising one or more types of bacteriophages in which each of the one or more types of bacteriophages bind to one or more specific types of receptor of one or more types of the bacteria if present in the sample to form complexes of bacteria and bacteriophage in which one or more types of bacteriophages are incapable of replicating inside said one or more types of bacteria; b) providing a second sample suspected of comprising one or more types of said bacteria in which the one or more types of bacteria are susceptible to infection by the one or more types of bacteriophage; c) contacting the first sample with the second sample to form a sample mixture in which the one or more types of bacteriophage if present in the first sample forms one or more complexes with the one or more types of bacteria in the sample mixture; d) incubating the sample mixture in a media comprising one or more bactericide or bacteriostatic agent; e) contacting one or more compounds to the media; f) measuring any change of the one or more compounds in presence of the media in which any change of one or more compounds is indicative of the presence or absence of one or more types of bacteriophage infected bacteria in the sample mixture, and the presence or absence of one or more types of bacteriophage infected bacteria in the sample mixture is indicative of the presence or absence of one or more types of bactericide or bacteriostatic agent resistant bacteria in the second sample.

In some embodiments, the one or more types of bacteriophages are immobilized on a solid support. In some embodiments, the complexes of bacteria and bacteriophage are immobilized on the solid support when one or more types of bacteriophages bind to one or more specific types of receptor of said one or more types of bacteria.

In some embodiments, the methods further include separating the complexes of the one or more types of bacteria and bacteriophage from the sample mixture and incubating the separated complexes in a media comprising one or more bactericide or bacteriostatic agent. In some embodiments, complexes of the bacteriophage and the bacteria are separated from the sample mixture, and the sample mixture depleted of the complexes are incubated in a media comprising one or more bactericide or bacteriostatic agent. In some embodiments, the methods include comparing a change of the one or more compounds in a media that is depleted of the complexes of the bacteriophage and bacteria with a change of the one or more compounds in a media comprising the complexes.

In some embodiments, the first sample comprising one or more types of bacteriophage is incubated with the second sample suspected of comprising one or more types of the bacteria prior to adding said one or more compounds. In some embodiments, the methods further include measuring any change of the one or more compounds in presence of said sample mixture over a period of time and determining the kinetic profile of the change of the one or more compounds in which the kinetic profile is indicative of the presence or absence of one or more types of bacteriophage infected bacteria in the sample mixture. The presence or absence of one or more types of bacteriophage infected bacteria in the sample mixture is indicative of the presence or absence of one or more types of bacteria in said second sample. In some embodiments, the methods further include comparing the kinetic profile of the change the one or more compounds with a kinetic profile of the change the one or more compounds in the presence of one or more types of bacteria susceptible to the one or more bactericide or bacteriostatic agent. A difference between kinetic profiles is indicative of the presence or absence of one or more types of bacteria resistant to one or more bactericide or bacteriostatic agent in said second sample.

In some embodiments, the one or more compounds is an enzymatic substrate of said one or more types of bacteria. In some embodiments, the one or more compounds is a redox compound. In some embodiments, the one or more compounds is Resazurin, Resorufin, Dihydroresorufin, or a combination thereof In some embodiments, the change the one or more compounds comprise a change in the oxidative state of Resazurin, Resorufin, Dihydroresorufin, or a combination thereof. In some embodiments, the measuring any change of the one or more compounds further comprises measuring the oxidative status of Resazurin, Resorufin, Dihydroresorufin or a combination thereof. In some embodiments, measuring the oxidative status of Resazurin, Resorufin, Dihydroresorufin or a combination thereof includes measuring the change in color, light scattering, fluorescence and/or absorbance intensity. In some embodiments, the method is carried out at about 20° C., 25° C., 27° C., 30° C., 32° C., 33° C., 35° C., 37° C., 40° C., 42° C., or 45° C.

In some embodiments, the one or more types of bacteriophage is chemically treated or genetically modified. In some embodiments, upon contacting the nucleic acid of one or more types of bacteriophages with a first compound inhibits bacteriophage replication in a bacterial cell. In some embodiments, the bacterial cell wall and/or cell membrane are intact. In some embodiments, the bacterial cell wall and/or cell membrane are not intact.

In some embodiments, the second compound preferentially binds to the nucleic acid of the bacteria after the formation of the complex of bacteriophage and bacteria. In some embodiments, the second compound preferentially binds to the nucleic acid of the bacteria prior to the formation of the complex of bacteriophage and bacteria. In some embodiments, the nucleic acid of one or more types of bacteriophage is dsDNA, ssDNA, ssRNA, or dsRNA. In some embodiments, the nucleic acid of the one or more types of bacteria is dsDNA or ssDNA.

In some embodiments, the first compound preferentially binds to dsDNA. In some embodiments, the first compound comprises a detectable label. In some embodiments, the first compound is reactive. In some embodiments, the first compound is photoreactive. In some embodiments, the first compound has low dissociation constant. In some embodiments, the first compound has high association constant. In some embodiments, the first compound binds to the nucleic acid of the bacteriophage and prevents the bacteriophage from replicating when inside the infected bacterial cell. Arresting the phage replication will prevent the bacteria from being lysed by the lytic phages while allowing the identification of the bacteria. In one example, the first compound is propidium monoazide, propidium iodide, ethidium bromide, or ethidium monoazide and/or a combination thereof. In another example, the bacteria containing the compound bound nucleic acid of the bacteriophage is viable. In some embodiments, the viable bacteria bound by the bacteriophage were detected. In some embodiments, the first compound is a cyanine based dye. In some embodiments, the detection further includes detection of the first compound.

In some embodiments, the second compound enters the bacteria. In some embodiments, the second compound binds to bacterial DNA. In some embodiments, the second compound comprises a detectable label. In some embodiments, the detectable label is a fluorescent moiety. In some embodiments, the second compound detects the cell activity/viability. In some embodiments, the second compound is an enzymatic substrate. In some embodiments, the second compound is a redox compound. In some embodiments, the second compound is Resazurin. In some embodiments, the modification of the second compound comprises reduction of Resazurin to Resorufin. In some embodiments, the detecting comprises measuring the fluorescence of Resorufin. In some embodiments, the detecting comprises measuring the fluorescence of Resorufin over time. In some embodiments, the modification of said second compound further comprises reduction of Resorufin to Dihydroresorufin. In some embodiments, the modification of said second compound further comprises oxidation of Dihydroresorufin to Resorufin. In some embodiments, the method is carried out at about 20° C., 25° C., 30° C., 32° C., 33° C., 35° C., 37° C., 40° C., 42° C., or 45° C. In some embodiments, the second compound is Calcein and/or Calcein-AM.

In one aspect, provided are kits for detecting the presence of bacteria in a sample. The kits include a) a first reagent comprising one or more types of virus in which each of the one or more types of viruses bind to one or more specific types of receptor of the one or more types of cell if present in the sample to form complexes of virus and cell in which the one or more types of viruses are incapable of replicating inside said one or more types of cell; b) a second reagent comprising one or more compounds preferentially interacts with one or more components of the cell in the presence of said one or more types of viruses of step (a); c) buffers; d) instruction for detecting the complexes by detecting the one or more compounds or modifications of the one or more compounds by the one or more types of cell in which detecting the complexes is indicative of the presence of the cell in the sample.

In some embodiments, the one or more types of cell are one or more types of bacteria, and one or more types of viruses are one or more types of bacteriophages that bind to one or more specific types of receptor of said one or more types of bacteria. In some embodiments, one or more types of bacteriophage are chemically treated or genetically modified. In some embodiments, one or more types of bacteriophage are treated with propidium monoazide or ethidium monoazide prior to contacting with said one or more types of bacteria. In some embodiments, one or more compounds is a redox reagent. In some embodiments, one or more compounds is an enzymatic substrate. In some embodiments, one or more compounds is Resazurin, Resorufin, Dihydroresorufin, or a combination thereof

In some embodiments, one or more types of bacteriophages are immobilized on a solid support. In some embodiments, the complexes of bacteria and bacteriophage are immobilized when one or more types of bacteriophages bind to one or more specific types of receptor of the one or more types of bacteria.

In one aspect, provided are methods of differentially labeling two nucleic acids. The methods include a) contacting a first nucleic acid with a first compound to generate a first nucleic acid having a first compound; b) contacting a second nucleic acid with a second compound in the presence of the first nucleic acid having a first compound in which the second compound preferentially binds to the second nucleic acid to generate a second nucleic acid having a second compound. The second compound is different from the first compound such that the first and second nucleic acids are differentially labeled with the first and second compounds, respectively.

In some embodiments, the first nucleic acid can be double stranded DNA (dsDNA), single stranded DNA (ssDNA), double stranded RNA (dsRNA), and/or single stranded RNA (ssRNA). In some embodiments, the first nucleic acid is genomic DNA. In some embodiments, the first nucleic acid is a bacteriophage genomic dsDNA or ssDNA. In some embodiments, the first nucleic acid is a bacteriophage genomic dsRNA or ssRNA. In some embodiments, the bacteriophage genomic DNA or RNA is present inside an intact bacteriophage in which the bacteriophage is capable of specifically binding to a bacterial receptor specific for the bacteriophage. In some embodiments, the second nucleic acid is dsDNA. In some embodiments, the second nucleic acid is genomic DNA. In some embodiments, the second nucleic acid is a bacterial genomic DNA.

In some embodiments, the first compound can be phenanthridine dye, acridine dye, indoles, or imidazole dyes. In some embodiments, in relation to the second compound, the first compound is a blocking compound, a non-interacting compound, or a displacing compound. In some embodiments, the first compound preferentially binds to ssDNA, ssRNA or dsRNA. In some embodiments, the first compound binds to the first nucleic acid and inhibits binding of other detectable labels from binding to the first nucleic acid.

In some embodiments, the second compound binds to ssDNA and/or dsDNA. In some embodiments, the second compound binds to ssRNA and/or dsRNA. In some embodiments, the second compound comprises a detectable label. In some embodiments, the detectable label comprises a fluorescent moiety. In some embodiments, the second compound has a dissociation constant that is same as the first compound. In some embodiments, the second compound has a dissociation constant that is more than the first compound. In some embodiments, the second compound does not interact with the first compound. In some embodiments, the second compound is not displaced by the first compound.

In one aspect, provided are methods of differentially labeling two proteins. The methods include a) contacting a first protein with a first compound to generate a first protein comprising a first compound; b) contacting a second protein with a second compound in the presence of the first protein comprising a first compound in which the second compound preferentially binds to the second protein to generate a second protein comprising a second compound. The second compound is different from the first compound such that the first and second proteins are differentially labeled with the first and second compounds, respectively.

In some embodiments, the first compound binds to a functional group of the first protein such as amine, carboxyl, phosphate, aldehyde, thiol, hydroxyl, or carbonyl. In some embodiments, the first compound binds to an amino acid, a group of amino acids or a peptide sequence of the first protein. In some embodiments, the first compound can be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, or alkyne. In some embodiments, the first compound binds to the first protein and inhibits binding of other compounds from binding to the first protein. In some embodiments, the first compound comprises a detectable label.

In some embodiments, the first protein is a bacteriophage protein. In some embodiments, the bacteriophage protein is a bacteriophage capsid protein. In some embodiments, the bacteriophage protein is part of an intact bacteriophage which is capable of specifically binding to a bacterial receptor specific for the bacteriophage. In some embodiments, the second protein is a bacterial protein.

In some embodiments, the second compound binds to a function group of the second protein such as amine, carboxyl, phosphate, aldehyde, thiol, hydroxyl, and carbonyl. In some embodiments, the second compound binds to an amino acid, a group of amino acids or a peptide sequence of the second protein. In some embodiments, the second compound comprises a detectable label. In some embodiments, the second compound has a dissociation constant same as the first compound. In some embodiments, the second compound has a dissociation constant more than the first compound. In some embodiments, the second compound does not interact with the first compound. In some embodiments, the second compound is not displaced by the first compound.

In some embodiments, the first and second compounds bind to the bacteriophage and bacteria, respectively by an interaction such as van der Waals interaction, electrostatic interaction, covalent interaction, hydrophilic interaction, hydrophobic interaction, hydrogen bonding interaction, ionic interaction, magnetic interaction, or a combination of the stated interactions. In some embodiments, the first compound is modified upon contacting said bacteriophage nucleic acid. In some embodiments, the second compound is modified upon binding to the nucleic acid of the bacteria. In some embodiments, the modification can be by forming a covalent bond, changing its composition, changing its electronic structure.

In some embodiments, the methods further include contacting the bacteriophage with a third compound in which the third compound binds to a bacteriophage protein. In some embodiments, the third compound binds to bacteriophage capsid protein. In some embodiments, the third compound comprises a detectable label. In some embodiments, the methods further include detecting the complex of bacteriophage and bacteria by detecting the detectable label of the third compound.

In some embodiments, the third compound binds to a function group of bacteriophage protein such as amine, carboxyl, phosphate, aldehyde, thiol, hydroxyl, or carbonyl. In some embodiments, the second compound binds to an amino acid, a group of amino acids or a peptide sequence of the bacteriophage protein. In some embodiments, the third compound can be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, or alkyne. In some embodiments, the third compound has a low dissociation constant. In some embodiments, the third compound binds to a bacteriophage protein and inhibits binding of other compounds from binding to the bacteriophage protein. In some embodiments, the third compound is an antibody.

In some embodiments, the methods further include contacting the bacteria with a fourth compound in which the fourth compound binds to the bacteria. In some embodiments, the fourth compound binds to a bacterial protein. In some embodiments, the fourth compound is an antibody. In some embodiments, the fourth compound is permeable to bacterial cell wall and bacterial cell membrane. In some embodiments, the fourth compound comprises a detectable label. In some embodiments, the methods further include detecting the complex of bacteriophage and bacteria by detecting the detectable label of the fourth compound. In some embodiments, the fourth compound has a dissociation constant same as the first, second, or third compounds. In some embodiments, the fourth compound has a dissociation constant more than the first, second, or third compounds. In some embodiments, the second compound does not interact with the first, second, or third compounds. In some embodiments, the second compound is not displaced by the first, second, or third compounds.

In some embodiments, the fourth compound binds to a functional group of bacterial protein such as amine, carboxyl, phosphate, aldehyde, thiol, hydroxyl, or carbonyl. In some embodiments, the fourth compound binds to an amino acid, a group of amino acids or a peptide sequence of the bacterial protein.

In some embodiments, the fourth compound can be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, or alkyne. In some embodiments, the fourth compound has a low dissociation constant. In some embodiments, the fourth compound preferentially binds to bacterial proteins. In some embodiments, the fourth compound binds to bacterial cell wall. In some embodiments, the fourth compound binds to the peptidoglycan of bacterial cell wall. In some embodiments, the fourth compound is gram stain. In some embodiments, the fourth compound binds to bacterial cell membrane.

In some embodiments, the first and second compounds are different from each other. In some embodiments, the third and fourth compounds are different from each other and from said first and second compounds. In some embodiments, the detection of the complex of bacteriophage and bacteria is by detecting two or more compounds.

In some embodiments, the bacteriophages can be replaced with receptor binding element of a bacteriophage in which the receptor binding element of the bacteriophage specifically binds to receptors of the bacteria. In some embodiments, the bacterial cell wall and/or cell membrane are intact. In some embodiments, the bacterial cell wall and/or cell membrane are not intact. In some embodiments, the bacterial cells are dormant. In some embodiments, the bacteriophage is present within the cells.

In some embodiments, the second compound preferentially binds to the protein of the bacteria after the formation of the complex of bacteriophage and bacteria. In some embodiments, the second compound preferentially binds to the protein of the bacteria prior to the formation of the complex of bacteriophage and bacteria.

In some embodiments, the first compound has a low dissociation constant. In some embodiments, the first compound is an antibody. In some embodiments, the first compound comprises a detectable label. In some embodiments, the first compound binds to a functional group of the bacteriophage protein such as amine, carboxyl, phosphate, aldehyde, thiol, hydroxyl, or carbonyl. In some embodiments, the first compound binds to an amino acid, a group of amino acids or a peptide sequence of the bacterial protein. In some embodiments, the first compound can be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, and alkyne. In some embodiments, the first compound binds to the bacteriophage protein and inhibits binding of other compounds from binding to the bacteriophage protein. In some embodiments, the bacteriophage protein is bacteriophage capsid protein.

In some embodiments, the second compound binds to a functional group of a bacterial protein selected from the group consisting of amine, carboxyl, phosphate, aldehyde, thiol, hydroxyl, or carbonyl. In some embodiments, the second compound binds to an amino acid, a group of amino acids or a peptide sequence of the bacterial protein. In some embodiments, the second compound is an antibody. In some embodiments, the second compound comprises a detectable label. In some embodiments, the detectable label comprises a fluorescent moiety. In some embodiments, the second compound has a dissociation constant same as the first compound. In some embodiments, the second compound has a dissociation constant more than the first compound. In some embodiments, the second compound does not interact with the first compound. In some embodiments, the second compound is not displaced by the first compound.

In some embodiments, the methods further include contacting the bacteriophage with a third compound in which the third compound binds to the bacteriophage nucleic acid. In some embodiments, the bacteriophage nucleic acid is dsDNA. In some embodiments, the third compound comprises a detectable label. In some embodiments, the methods further include detecting the complex of bacteriophage and bacteria by detecting the detectable label of the third compound. In some embodiments, the third compound has a low dissociation constant. In some embodiments, the third compound binds to the bacteriophage nucleic acid and inhibits binding of other compounds from binding to the bacteriophage nucleic acid. In some embodiments, the third compound binds to the nucleic acid of said bacteriophage and prevents the bacteriophage from replicating when inside the infected bacterial cell. Arresting the phage replication will prevent the bacteria from being lysed by the lytic phages while allowing the identification of the bacteria. In one example, the first compound is propidium monoazide, propidium iodide, ethidium bromide, or ethidium monoazide. In another example, the bacteria containing the compound bound nucleic acid of the bacteriophage is viable. In some embodiment, the viable bacteria bound by the bacteriophage were detected.

In some embodiments, the methods further include contacting the bacteria with a fourth compound in which the fourth compound binds to the bacteria. In some embodiments, the fourth compound binds to bacterial protein. In some embodiments, the fourth compound is an antibody. In some embodiments, the fourth compound is permeable to bacterial cell wall and bacterial cell membrane. In some embodiments, the fourth compound binds to nucleic acid of said bacteria. In some embodiments, the fourth compound comprises a detectable label. In some embodiments, the detectable label comprises a fluorescent moiety.

In some embodiments, the fourth compound binds to a functional group of a protein of the bacteria such as amine, carboxyl, phosphate, aldehyde, thiol, hydroxyl, or carbonyl. In some embodiments, the fourth compound can be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, or alkyne. In some embodiments, the fourth compound has a low dissociation constant. In some embodiments, the fourth compound preferentially binds to bacterial proteins. In some embodiments, the fourth compound binds to bacterial cell wall. In some embodiments, the fourth compound binds to the peptidoglycan of bacterial cell wall. In some embodiments, the fourth compound is gram stain. In some embodiments, the fourth compound binds to bacterial cell membrane.

In some embodiments, the first and second compounds are different from each other. In some embodiments, the third and fourth compounds are different from each other and from said first and second compounds. In some embodiments, the detection of the complex of bacteriophage and bacteria is by detecting two or more compounds.

In some embodiments, upon association with the nucleic acid of the bacteriophage, the first detectable label inhibits binding of the other detectable labels to the bacteriophage nucleic acid. In some embodiments, the first compound is photoreactive. In some embodiments, the first compound comprises a detectable label. In some embodiments, the detectable label comprises a fluorescent moiety. In some embodiments, the first compound is propidium monoazide or ethidium monoazide. In some embodiments, the first compound is a cyanine based dye. In some embodiments, the first compound is propidium iodide or ethidium bromide. In some embodiments, the first compound binds to the nucleic acid of said bacteriophage and prevents the bacteriophage from replicating when inside the infected bacterial cell. In one example, the first compound is propidium monoazide, propidium iodide, ethidium bromide, or ethidium monoazide. In another example, the bacteria containing the compound bound to the nucleic acid of the bacteriophage is viable. In some embodiment, the viable bacteria bound by the bacteriophage were detected. Arresting the phage replication will prevent the bacteria from being lysed by the lytic phages while allowing the identification of the bacteria. In some embodiments, the first compound binds to bacteriophage protein. In some embodiments, the bacteriophage protein is bacteriophage capsid protein. In some embodiments, the first compound can be phenanthridine dye, acridine dye, indoles, or imidazole dyes.

In some embodiments, the second compound binds to bacterial protein. In some embodiments, the second compound is an antibody. In some embodiments, the second compound is permeable to bacterial cell wall and bacterial cell membrane. In some embodiments, the second compound binds to the nucleic acid of the bacteria. In some embodiments, the nucleic acid is dsDNA. In some embodiments, the second compound comprises a detectable label. In some embodiments, the detectable label comprises a fluorescent moiety.

In some embodiments, the first and/or second compounds bind to the bacteriophage and bacteria, respectively by an interaction such as van der Waals interaction, electrostatic interaction, covalent interaction, hydrophilic interaction, hydrophobic interaction, hydrogen boding interaction, ionic interaction, magnetic interaction or a combination of the stated interactions. In some embodiments, the first and/or second compounds are modified upon association with said bacteriophage. In some embodiments, the modification is by a method for example, forming a covalent bond, changing its composition, or changing its electronic structure.

In some embodiments, the second compound preferentially binds to said bacteria after the formation of the complex of bacteriophage and bacteria. In some embodiments, the second compound preferentially binds to the bacteria prior to the formation of the complex of bacteriophage and bacteria.

In one aspect, provided are methods for detecting the presence of bacteria in a sample. The methods include a) providing a receptor binding element of a bacteriophage in which the receptor binding element of the bacteriophage specifically binds to receptors of the bacteria; b) contacting the sample suspected of comprising the bacteria with the receptor binding element of the bacteriophage and a detectable label. The receptor binding element of the bacteriophage binds specifically to the receptors of the bacteria if present in the sample to form a complex; c) detecting the complex in which the complex is indicative of the presence of the bacteria in the sample. In some embodiments, the bacterial cell wall and/or cell membrane are intact. In some embodiments, the bacterial cell wall and/or cell membrane are not intact.

In some embodiments, the sample suspected of being contaminated by bacteria is partitioned into solid phase and liquid phase prior to contacting with a bacteriophage or a receptor binding element of the bacteriophage in which the partitioned solid or liquid phase of the sample is contacted with the bacteriophage or a receptor binding element of the bacteriophage. In some embodiments, the partitioning of the sample into solid and liquid phases is by centrifugation. In some embodiments, the partitioning of the sample into solid and liquid phases is by filtration. In some embodiments, the partitioning of the sample into solid and liquid phases is by changing the pH. In some embodiments, the partitioning of the sample into solid and liquid phases is by a combination of changing the pH and filtration. In some embodiments, the partitioning of the sample into solid and liquid phases is by a combination of changing the pH and centrifugation. In some embodiments, the partitioning of the sample into solid and liquid phases is by a combination of changing the pH, centrifugation, and filtration. In some embodiments, the changing of pH is by lowering the pH. In some embodiments, the changing of pH is by increasing the pH.

In some embodiments, the bacteria in the sample are concentrated prior to contacting with bacteriophage or a receptor binding element of the bacteriophage. In some embodiments, the concentration of the bacteria is by changing the pH. In some embodiments, the concentration of the bacteria is by salt concentration. In some embodiments, the concentration of the bacteria is by centrifugation. In some embodiments, the concentration of the bacteria is by filtration. In some embodiments, the filtration is by membrane filtration. In some embodiments, the pore size of the membrane is about 10 nm to about 1000 nm. In some embodiments, the concentration of the bacteria is by changing the ionic strength of the sample. In some embodiments, the concentration of the bacteria is by changing the pH of the sample. In some embodiments, the changing of pH is by lowering the pH. In some embodiments, the changing of pH is by increasing the pH.

In some embodiments, the methods further include removing the complex of bacteriophage or a receptor binding element of the bacteriophage and bacteria from the mixture of bacteriophage or receptor binding element of the bacteriophage and the sample to generate a clarified sample and detecting the presence of free unbound bacteria in the clarified sample, in which the presence of the free unbound bacteria is indicative of bacteria that are resistant to bacteriophage infection in a sample.

In some embodiments, the methods further include separating the complex of the bacteria and the bacteriophage or the receptor binding element of the bacteriophage from the sample prior to detection of the complex. In some embodiments, the separation of the complex is by applying an electric field. In some embodiments, the separation is by magnetic field. In some embodiments, the separation of the complex is by size. In some embodiments, the separation is by centrifugation. In some embodiments, the separation is by filtration. In some embodiments, the filtration is by membrane filtration. In some embodiments, the pore size of said membrane is about 1 nm to about 1 μm. In some embodiments, the complex is based on the difference in density of said complex, said bacteria, and said bacteriophage. In some embodiments, the separation of the complex is changing the pH, ionic strength, viscosity, temperature or pressure. In some embodiments, the separation of the complex is by addition of a surfactant, a competitive molecule, an enzyme, and/or carbohydrates.

In some embodiments, the bacteriophage or the receptor binding element of the bacteriophage is immobilized on a solid support. In some embodiments, upon contacting of the sample suspected of comprising the bacteria with the immobilized bacteriophage or the immobilized receptor binding element of the bacteriophage, at least a portion of said bacteria specifically binds to said immobilized bacteriophage or said receptor binding element of said bacteriophage to form said complex, and at least a portion of the complexes are immobilized on the solid support.

In some embodiments, at least a portion of the complex immobilized on the solid support is separated from the sample prior to detection of the detectable labels. In some embodiments, the bacteriophage or the receptor binding element of the bacteriophage comprises a first member of a binding pair and the solid support comprises a second member of the binding pair. Binding of the first and second members of the binding pair immobilizes the bacteriophage or the receptor binding element of the bacteriophage to the solid support.

In some embodiments, the bacteriophage or the receptor binding element of the bacteriophage comprises a tag and the solid support comprises an antibody that specifically binds to the tag in which the tag comprises a first member of a binding pair and the antibody comprises a second member of the binding pair. In some embodiments, the tag is selected from the group consisting of His6, FLAG®, GST, MBP, myc. In some embodiments, the binding pair is biotin and streptavidin.

In some embodiments, the bacteriophage or the receptor binding element of the bacteriophage is immobilized on the solid support by an interaction such as covalent interaction, electrostatic interaction, hydrogen bond, hydrophobic interaction, hydrophilic interaction, or the combination of the above interactions.

In some embodiments, the solid surface comprises functional groups for immobilization by covalent bond. In some embodiments, the functional groups can be poly L-lysine, aminosilane, epoxysilane, aldehydes, amino groups, epoxy groups, cyano groups, ethylenic groups, carboxylic groups, hydroxyl groups, thiol groups, N-hydroxysuccinimide (NHS), azide, alkyne, maleimide, or streptavidin.

In some embodiments, the solid support is a bead. In some embodiments, the bead is a magnetic bead. In some embodiments, the bead is a streptavidin bead. In some embodiments, the bead is a fluorescent bead. In some embodiments, the methods may include using plurality of fluorescent beads. In some embodiments, the bead comprises antibody.

In some embodiments, the solid support is a microarray. In some embodiments, the solid support is a fluidic device. In some embodiments, the solid support is a flow cell. In some embodiments, the solid support is a fluidic cartridge. In some embodiments, the solid support comprises metal coated surface. In some embodiments, the solid support comprises dielectric coated glass. In some embodiments, the solid support comprises nanowells, nanochannels or nanostructures. In some embodiments, the solid support comprises of porous material. In some embodiments, the solid support further comprises a flow cell in which the bacteriophage or the receptor binding element of the bacteriophage is immobilized on the solid support, and the bacteria is in a liquid communication with the bacteriophage or the receptor binding element of said bacteriophage.

In some embodiments, the solid support comprises a sensor. In some embodiments, the sensor comprises electrodes in which the detection of the complex further comprises detecting a change in electrical signal. In some embodiments, a voltage sensitive detectable label is used in which a complex is detected by a change in electrical signal once the complex is formed.

In some embodiments, the detection of the complex comprises detecting a change in mass on said solid support. In some embodiments, the detection of the complex comprises detecting a change in resonance frequency of a sensor. In some embodiments, the sensor is a piezoelectric sensor in which the detection of the complex further comprises detecting a change in dissipation of shear movement of the sensor. In some embodiments, the detection of the complex comprises detecting surface Plasmon resonance. In some embodiments, the detection of the complex comprises detection by scanning probe microscopy. In some embodiments, the detection is by atomic force microscopy. In some embodiments, the detection of the complex is by Raman spectroscopy. In some embodiments, the detection of the complex is by surface enhanced Raman spectroscopy. In some embodiments, an illuminating light or laser source has a wavelength of about 200nm to about 2000nm. In some embodiments, the detection of the complex comprises detection by fluorescence spectroscopy. In some embodiments, the detection of the complex comprises detection by electron microscopy. In some embodiments, the detection of said complex comprises detecting a change in heat flow due to the formation of the complex. In some embodiments, the detection is by a calorimeter. In some embodiments, the calorimeter is an isothermal calorimeter. In some embodiments, the detection of the complex comprises detecting the forward and sideward scattering of the light by the formation of the complex. In some embodiments, the detection is by a flow cytometer.

In some embodiments, the complex is detected by detecting the nucleic acid of the bacteria. In some embodiments, the detection of the nucleic acid is by mass spectrometry. In some embodiments, the nucleic acid of said bacteria is detected by electrophoresis. In some embodiments, the complex is detected by electrophoresis. In some embodiments, electrophoresis is done by extracting the DNA. In some embodiments, the electrophoresis can be gel pulse electrophoresis. In some embodiments, the information about the bacteria is deduced by looking at a DNA band. In some embodiments, the information about the bacteria is deduced by electrophoresis of restriction digested DNA fragments.

In some embodiments, the bacteria are pathogenic to plants. In some embodiments, the bacteria are pathogenic to crop.

In some embodiments, the bacteria are pathogenic to animals. In some embodiments, the bacteria are pathogenic to vertebrates. In some embodiments, the bacteria are pathogenic to fish. In some embodiments, the bacteria are pathogenic to mammal. In some embodiments, the bacteria are pathogenic to human. In some embodiments, the bacteria are pathogenic to non-human animal. In some embodiments, the bacteria are pathogenic to farm animals. In some embodiments, the bacteria are pathogenic to dairy animals. In some embodiments, the bacteria are pathogenic to chickens. In some embodiments, the bacteria are pathogenic to wild animals. In some embodiments, the bacteria are pathogenic to marine animals. In some embodiments, the bacteria are pathogenic to domesticated animals.

In some embodiments, the bacteria are pathogenic to non-vertebrate animals. In some embodiments, the bacteria are pathogenic to bacteria are pathogenic to insects. Non-limiting examples include bees, butterflies, and mosquitoes.

In some embodiments, the bacteria are Gram negative bacteria. Non-limiting examples of gram negative bacteria include Citrobacter freundii, Klebsiella pneumoniae, Acinetobacter, Proteus mirabilis, Pseudomonas aeruginosa, Salmonella enteritidi, and Escherichia coli. In some embodiments, the bacteria are gram positive bacteria. Non-limiting examples of gram positive bacteria include Staphylococcus aureus and Enterococci. In some embodiments, the bacteria are neither gram positive nor gram negative bacteria.

In some embodiments, the families of said bacteriophage that can infect bacteria used in dairy or wine fermentation include Myoviridae, Siphoviridae and Podoviridae.

In some embodiments, the bacteria, the bacteriophage, and/or or the receptor binding element of the bacteriophage comprise more than one detectable label. In some embodiments, the detectable labels are different. In some embodiments, one detectable label is associated with the nucleic acid of the bacteria and a second detectable label is associated with the bacterial proteins. In some embodiments, one detectable label is associated with the nucleic acid of the bacteriophage and a second detectable label is associated with the bacteriophage proteins.

In some embodiments, the detectable label comprises a fluorescent moiety. In some embodiments, the detection of the complex is by detecting the fluorescence signal from said fluorescent moiety.

In some embodiments, the detectable label is associated with the bacteria or the bacteriophage by van der Waals interaction, electrostatic interaction, hydrogen boding interactions, ionic interaction, hydrophobic interactions, hydrophilic interactions, magnetic interaction, covalent interaction, or a combination thereof. In some embodiments, the detectable label is modified upon association with said bacteria or bacteriophage. In some embodiments, the modification is by forming a covalent bond. In some embodiments, the modification is by changing the composition (chemical change such as protonation/de-protonation), changing its electronic structure (oxidation/reduction) of the detectable label.

In one aspect, provided are methods of diagnosing an individual suspected of having bacterial infection. The methods include: a) providing a sample from the individual; b) detecting the presence of bacteria in the sample using any one of the above described methods. Detecting the complex is indicative of bacterial infection in the individual.

In one aspect, provided are methods of prognosis of bacterial infection in an individual. The methods include: a) providing samples from the individual at two or more time intervals; b) detecting the presence of bacteria in the samples using any one of the above described methods. A relative increase in the amount of complex detected at later time points is indicative of the progression of bacterial infection in the individual, and a relative decrease in the amount of complex detected at later time points is indicative of the remission of bacterial infection in the individual.

In some embodiments, one or more of the compounds are permeable to bacterial cell wall and bacterial cell membrane. In some embodiments, one or more of the compounds bind to bacteriophage capsid protein. In some embodiments, one or more of the compounds bind to a bacterial protein. In some embodiments, one or more of the compounds bind to the peptidoglycan of bacterial cell wall. In some embodiments, one or more of the compounds are modified upon binding to the nucleic acid or protein of the bacteriophage or bacteria. In some embodiments, the modification can be by forming a covalent bond, changing its composition, changing its electronic structure. In some embodiments, one or more of the compounds is not displaced by another compound.

In one aspect, provided are methods of detecting the presence of bacteriophage in a sample. The methods include a) providing a sample suspected of comprising the bacteriophage, wherein the bacteriophage specifically binds to receptors of specific bacteria to form a complex of bacteriophage and bacteria; b) providing bacterial receptors specific for the bacteriophage; c) contacting the sample suspected of comprising the bacteriophage with the bacterial receptors and a detectable label, wherein the bacteriophage binds specifically to the bacterial receptors of the bacteria to form a complex, and wherein the detectable label preferentially binds to the bacteriophage; and d) detecting the complex by detecting the detectable label, wherein the complex is indicative of the presence of the bacteriophage in the sample.

In some embodiments, the detectable label binds to the nucleic acid of the bacteriophage. In some embodiments, the detectable label binds to the protein of the bacteriophage. In some embodiments, the protein is bacteriophage capsid protein. In some embodiments, the detectable label comprises a fluorescent moiety.

In some embodiments, the receptors of the bacteria are immobilized on a solid support. In some embodiments, the solid surface comprises functional groups for immobilization by covalent bond. In some embodiments, the functional groups are selected from the group consisting of poly L-lysine, aminosilane, epoxysilane, aldehydes, amino groups, epoxy groups, cyano groups, ethylenic groups, carboxylic groups, hydroxyl groups, thiol groups, N-hydroxysuccinimide (NHS), azide, alkyne, maleimide, and streptavidin.

In some embodiments, the solid support is a bead. In some embodiments, the bead is a magnetic bead. In some embodiments, the bead is a fluorescent bead. In some embodiments, the bead comprises antibody. In some embodiments, the bead is a streptavidin bead. In some embodiments, the solid support is a microarray. In some embodiments, the solid support is a fluidic device. In some embodiments, the solid support is a flow cell. In some embodiments, the solid support is a fluidic cartridge. In some embodiments, the solid support comprises metal coated surface. In some embodiments, the solid support comprises dielectric coated glass. In some embodiments, the solid support comprises nanowells, nanochannels or nanostructures. In some embodiments, the solid support comprises of porous material. In some embodiments, the solid support comprises a sensor.

In some embodiments, the bacterial receptors are cell-free bacterial receptors. In some embodiments, the bacterial receptors are purified bacterial receptors. In some embodiments, the bacterial receptors are recombinant bacterial proteins. In some embodiments, the bacterial receptors are part of a bacterial cell wall or a bacterial cell membrane fragment. In some embodiments, the bacterial receptors are naturally occurring bacterial receptors derived from bacterial cell. In some embodiments, the bacterial receptors are chemically modified. In some embodiments, the bacterial receptors comprise a detectable label. In some embodiments, the detectable label of said bacterial receptors is a fluorescence moiety. In some embodiments, the method further comprises detecting the detectable label of said bacterial receptors, and wherein detecting the detectable label of said bacterial receptors is indicative of the presence of said bacteriophage in said sample.

In one aspect, provided are methods for detecting the presence of bacteria in a sample. The methods include a) providing a bacteriophage in which the bacteriophage specifically binds to receptors of the bacteria to form a complex; b) incubating the bacteriophage with the sample; c) providing a detectable label; d) measuring the signal from the detectable label over time in which the kinetic profile of the detectable label is indicative of the presence or absence of phage infected bacteria. In some embodiments, the detectable label is Resazurin, Resorufin, Dihydroresorufin, or a combination thereof. In some embodiments, the detectable label is Resorufin or Dihydroresorufin. In some embodiments, the Resazurin is reduced to Resorufin. The Resazurin (weakly fluorescent) gets reduced to fluorescent Resorufin due to metabolic activity of the cell. This reduction is irreversible. In some embodiments, Resorufin is further reduced by the cell to Dihydroresorufin (non-fluorescent). This reduction is reversible. The inventors have found that the equilibrium between Resorufin and Dihydroresorufin is significantly affected by the presence of phage in the sample. In the case of phage infected cells, the Dihydroresorufin is preferably oxidized back to Resorufin while in the case of uninfected cells; the Dihydroresorufin is a favored product in the sample mixture. Also, inventors of the present application have surprisingly and unexpectedly found that in the case of phage infected cells, the reduction of Resazurin to Resorufin is slower than that of the uninfected cells. Thus, the fluorescence intensity of Resorufin appears faster for non-infected cells as compared to phage infected cells. In some embodiments, in the case of phage infected cells, the absorbance of light and/or fluorescent intensity of Resorufin does not decrease as reduction to Dihydroresorufin is not favored. In some embodiments, the Resorufin increases in intensity with time in comparison to Dihydroresorufin as Dihydroresorufin is oxidized back to Resorufin. In contrast, in the case of uninfected cells the absorbance of light and/or fluorescent intensity of Resorufin diminishes with time as Resorufin is further reduced to Dihydroresorufin and does not reappear or reappears at a significantly later time than in the case of phage infected cells.

The inventors of the present application have employed this unexpected finding in detecting the presence of bacteria in a sample. In some embodiments, detection includes determining the change in the color intensity, absorbance, scattering of light, fluorescence intensity, change in the slope of the fluorescence kinetic profile of Resazurin to Resorufin and/or Resorufin to Dihydroresorufin reduction by the phage infected bacteria. In some embodiments, the methods further include comparing the kinetic profile of Resazurin to Resorufin reduction of the sample with that of the uninfected bacteria in which a shift in the kinetic peak is indicative of the presence of phage infected bacteria in the sample. Additionally, the diminution of the Resorufin absorbance of light and/or fluorescent intensity with time and reappearance of its fluorescence intensity is indicative of phage infected bacteria and thus indicative of the bacteria in a sample. In contrast, a diminution of the Resorufin absorbance of light and/or fluorescent intensity without its reappearance in a significant amount of time is indicative of uninfected bacteria and thus absence of bacteriophage in a sample.

In some embodiments, the bacterial cells susceptible to infection by the bacteriophage are incubated with samples suspected of comprising bacteriophage to form a complex if bacteria are present in a sample, prior to adding Resazurin to the incubation mixture. In some embodiments, Resazurin is replaced by adding Resorufin to the incubation mixture. In some embodiments Resorufin is replaced by adding Dihydroresorufin to the incubation mixture. In some embodiments, the sample suspected of comprising bacterial cells and susceptible to infection by the bacteriophage are incubated with bacteriophages for at least about 1, 2, 3, 5, 7, 10, 15, 20, 25, 30, 35, 40, 45, 50, 60, 65, 70, 75, 80, 90 or more minutes prior to adding Resazurin and/or Resorufin and/or Dihydroresorufin to the incubation mixture. In some embodiments, Resazurin and/or Resorufin and/or Dihydroresorufin is added at the time of incubation. In some embodiments, the incubation is carried out at about 20° C., 25° C., 27° C., 30° C., 32° C., 35° C., 37 or 40° C.

In some embodiments, the fluorescence of Resorufin is measured at about 550 nm to about 580 nm. In some embodiments, the fluorescence of Resorufin is measured at about 570 nm, 572 nm, 573 nm, 575 nm, 577 nm, or 580 nm or higher wavelength. In some embodiments, the fluorescence of Resorufin is measured for about 2, 5, 10, 15, 20, 25, 30, 35, 40, 45, 50, 55, 60, 65, 70, 75, 80, 85, 90, 95, 100, 105, 110, 115, 120, 125, 130, 135, 140 or more minutes after adding Resazurin and/or Resorufin and/or Dihydroresorufin to the incubation mixture of bacteriophage and samples suspected of comprising bacteria.

In one aspect, provided are methods of determining the susceptibility of bacteria to a bactericide agent or bacteriostatic agent in a sample. The methods include a) providing a sample comprising bacteria; b) providing a bactericide or bacteriostatic agent; c) incubating said sample with the bactericide or bacteriostatic agent; d) providing a detectable label; e) determining the changing color, absorbance, scattering of light, fluorescence signal, kinetic profile of the detectable label over time in which the kinetic profile of the detectable label is indicative of the susceptibility of the bacteria to the bactericide or bacteriostatic agent. In some embodiments, the detectable label measures cell viability. In some embodiments, the detectable label is Resazurin. In some embodiments, the detectable label is Resorufin and/or Dihydroresorufin. In some embodiments, the detectable label is Resazurin and/or Resorufin and/or Dihydroresorufin. In some embodiments, the Resazurin is reduced by the bacteria to Resorufin. In some embodiments, Resorufin is further reduced by the cell to Dihydroresorufin (non-fluorescent). This reduction is reversible. The inventors have found that the equilibrium between Resorufin and Dihydroresorufin is significantly affected by the presence of a bactericide or bacteriostatic agent in the sample comprising bacteria susceptible to the bactericide or bacteriostatic agent. In such a case, the Dihydroresorufin is preferably oxidized back to Resorufin while in the case of bacterial cells not susceptible to the bactericide or bacteriostatic agent or in the absence of the bactericide or bacteriostatic agent; the Dihydroresorufin is a favored product in the sample mixture. Also, inventors of the present application have surprisingly and unexpectedly found that in the case of bacterial cells susceptible to the bactericide or bacteriostatic agent, the reduction of Resazurin to Resorufin is slower in the presence of the bactericide or bacteriostatic agent than that of the non-susceptible bacterial cells or the bacterial cells in the absence of the bactericide or bacteriostatic agent. Thus, the fluorescence intensity of Resorufin appears faster for the non-susceptible bacterial cells or the bacterial cells in the absence of the bactericide or bacteriostatic agent as compared to susceptible bacterial cells in the presence of the bactericide or bacteriostatic agent. In some embodiments, in the case of susceptible bacterial cells in the presence of the bactericide or bacteriostatic agent, the absorbance, scattering and/or fluorescence of Resazurin does not decrease, as reduction to Dihydroresorufin is not favored. In some embodiments, in the case of susceptible bacterial cells in the presence of the bactericide or bacteriostatic agent, the absorbance, scattering and/or fluorescent intensity of Resorufin does not decrease as reduction to Dihydroresorufin is not favored. In some embodiments, the Resorufin increases in intensity with time in comparison to Dihydroresorufin as Dihydroresorufin is oxidized back to Resorufin. In contrast, in the case of the non-susceptible bacterial cells or the bacterial cells in the absence of the bactericide or bacteriostatic agent the absorbance, scattering and/or fluorescent intensity of Resorufin diminishes with time as Resorufin is further reduced to Dihydroresorufin and does not reappear or reappears at a significantly later time than in the case of susceptible bacterial cells in the presence of the bactericide or bacteriostatic agent.

The inventors of the present application have employed this unexpected finding in detecting the presence of bacterial cells susceptible to the bactericide or bacteriostatic agent in a sample. In some embodiments, detection includes determining the change in the color intensity, fluorescence intensity, change in the slope of the fluorescence kinetic profile of substrate, in this case, Resazurin to Resorufin and/or Resorufin to Dihydroresorufin reduction by the bacteria. In some embodiments, the methods further include comparing the kinetic profile of Resazurin to Resorufin reduction by non-susceptible bacterial cells or the bacterial cells in the absence of the bactericide or bacteriostatic agent. A shift in the kinetic peak is indicative of the presence of bacteria that is susceptible to bacteriostatic or bactericidal agent in the sample. Additionally, the diminution of the Resorufin absorbance, scattering and/or fluorescent intensity with time and reappearance of its fluorescence intensity is indicative of bacteria that is susceptible to bacteriostatic or bactericidal agent in the sample in a sample. In contrast, a diminution of the Resorufin absorbance and/or fluorescent intensity without its reappearance in a significant amount of time is indicative of non-susceptible bacterial cells or the bacterial cells in the absence of the bactericide or bacteriostatic agent in a sample.

In some embodiments, the sample comprising bacterial cells are incubated prior to adding Resazurin to the incubation mixture. In some embodiments, Resazurin is replaced by adding Resorufin to the incubation mixture. In some embodiments Resorufin is replaced by adding Dihydroresorufin to the incubation mixture. In some embodiments, the sample suspected of comprising bacterial cells and are incubated with bacteriophages for at least about 1, 2, 3, 5, 7, 10, 15, 20, 25, 30, 35, 40, 45, 50, 60, 65, 70, 75, 80, 90 or more minutes prior to adding Resazurin and/or Resorufin and/or Dihydroresorufin to the incubation mixture. In some embodiments, Resazurin and/or Resorufin and/or Dihydroresorufin is added at the time of incubation. In some embodiments, the incubation is carried out at about 20° C., 25° C., 27° C., 30° C., 32° C., 35° C., 37 or 40° C.

In some embodiments, the fluorescence of Resorufin is measured at about 570 nm, 572 nm, 573 nm, 575 nm, 577 nm, or 580 nm or higher wavelength. In some embodiments, the fluorescence of Resorufin is measured for about 2, 5, 10, 15, 20, 25, 30, 35, 40, 45, 50, 55, 60, 65, 70, 75, 80, 85, 90, 95, 100, 105, 110, 115, 120, 125, 130, 135, 140 or more minutes after adding Resazurin and/or Resorufin and/or Dihydroresorufin to the incubation.

In some embodiments, the methods and compositions of the present application are useful in detecting bacteria in a sample. In some embodiments, the methods and compositions of the present application are useful in detecting bacteria susceptible to a bactericide or a bacteriostatic agent in a sample. In some embodiments, the bacteria are lactic acid bacteria contamination in a yeast fermentation sample. In some embodiments, the sample is a food. In some embodiments, the sample can be from a patient suspected of having a bacterial infection.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A depicts bacteriophage directly immobilized on a solid support. FIG. 1B depicts a complex of bacteriophage and the bacteria immobilized on a solid support though the immobilized phage of FIG. 1A.

FIG. 2A depicts bacteriophage immobilized on a solid support through a linker. FIG. 2B depicts a complex of bacteriophage and the bacteria immobilized on a solid support though the immobilized phage of FIG. 2A.

FIG. 3(i) shows fluorescence image of SYBR Green dye stained S1 bacteria added to different beads shown in the schematic. FIG. 3(ii) shows the bright field image of the corresponding beads in the top panel. J1 bacteriophages are stained with PMA.

FIG. 4(i) shows fluorescence image of SYBR Green dye stained S1 bacteria added to beads comprising bacteriophage J1 immobilized through linkers as shown in the schematic. FIG. 4(ii) shows the bright field image of the corresponding beads in the top panel.

FIG. 5(i) shows fluorescence image of SYBR Green dye stained S1 bacteria added to beads comprising linkers without bacteriophage J1 as shown in the schematic. FIG. 5(ii) shows the bright field image of the corresponding beads in the top panel.

FIG. 6 shows an exemplary excitation and emission spectra of SYTOX® orange and propidium iodide.

FIG. 7 shows a schematic of detection of bacteriophage using immobilized bacteria on a solid support.

FIGS. 8A-C show the kinetic profile of the reduction of Resazurin to Resorufin to Dihydroresorufin by phage infected and uninfected bacterial cells.

FIGS. 9A-B show the kinetic profile of the reduction of Resazurin to Resorufin to Dihydroresorufin by phage infected and uninfected bacterial cells.

FIG. 10 show the kinetic profile of the reduction of Resazurin to Resorufin to Dihydroresorufin by phage infected and uninfected bacterial cells.

FIG. 11 show the kinetic profile of the reduction of Resazurin to Resorufin to Dihydroresorufin by phage infected and uninfected bacterial cells.

FIGS. 12A-B show the kinetic profile of the reduction of Resazurin to Resorufin to Dihydroresorufin and Resorufin to Dihydroresorufin by phage infected and uninfected bacterial cells.

FIGS. 13A-D show the kinetic profile of the reduction of Resazurin and Resorufin by phage infected and uninfected bacterial cells.

FIGS. 14A-D shows the non-lytic activity of PMA treated and lytic activity of bacteriophages without PMA. The strains of bacteriophages used are DT1 and T7.

FIGS. 15A-C Resazurin assay with E. coli with PMA, E. coli with T7, and E. coli.

FIG. 16. Cell viability assay of E. coli captured by PMA treated T7 containing beads. The beads were incubated with E. coli and Streptococcus thermophilus as a negative control.

FIG. 17 shows Biotinylated T7 magnetic beads used to test the capture of E. coli and antibiotic resistant E. coli (ER2508) and test for antibiotic resistance.

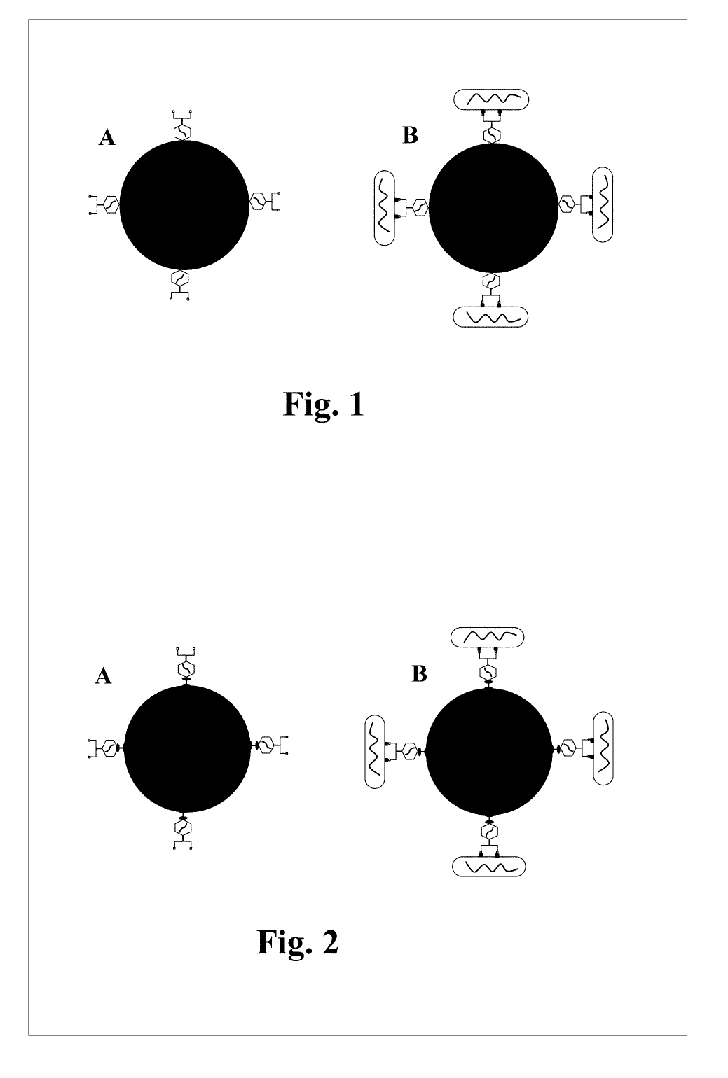

### DETAILED DESCRIPTION OF THE INVENTION

As used herein the term “change” or “modification” of a compound refers to the change in the physical, chemical, or oxidative state of the compound. In some embodiments, the change comprises covalent modification. In some embodiments, the modification is by forming a covalent bond. In some embodiments, the change is by changing the composition (chemical change such as protonation/de-protonation), changing its electronic structure (oxidation/reduction) of the detectable label. In some embodiments, a compound comprising a detectable label is modified upon association with bacteria or bacteriophage.

The term “surface plasmon resonance,” as used herein, refers to an optical phenomenon that allows for the analysis of real-time biospecific interactions by detection of alterations in concentrations within a biosensor matrix, for example using the BIACORE™ system (Pharmacia Biosensor AB, Uppsala, Sweden and Piscataway, N.J.).

As used herein the term “at least a portion” and/or grammatical equivalents thereof can refer to any fraction of a whole amount. For example, “at least a portion” can refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 99.9% or 100% of a whole amount.

As used herein the term “about” means +/−10%.

Solid Support

Solid support can be two-or three-dimensional and can comprise a planar surface (e.g., a glass slide) or can be shaped. A solid support can include glass (e.g., controlled pore glass (CPG)), corrugated glass, quartz, plastic (such as polystyrene (low cross-linked and high cross-linked polystyrene), polycarbonate, polypropylene and poly(methylmethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional solid support includes, for example, spheres, microparticles, beads, membranes, slides, plates, micromachined chips, nanoengineered chips tubes (e.g., capillary tubes), microwells, nanowells, microfluidic devices, nanofluidic devices, flow cells, channels, filters, fluidic cartridge. Solid support can include planar arrays or matrices capable of having regions that include populations of bacteriophage or bacteria.

In some embodiments, the solid support or its surface is non-planar, such as the inner or outer surface of a tube or vessel. In some embodiments, the solid support is a surface of a flow cell.

In some embodiments, solid support may include Surface Enhanced Raman Spectroscopy (SERS) surface. In some embodiments, solid support may include a base support and a metallic nanostructure layer deposited upon the base support. The nanomaterial employed in the nanostructure layer may be any SERS-active metallic material, such as silver, gold, copper, platinum, titanium, chromium, combinations thereof or the like. In addition, the nanostructure layer may assume any form, such as nanoparticles, nanoaggregates, nanopores/nanodisks, nanorods, nanowires, or combinations thereof. In some embodiments, the nanoparticles are arranged on the base support by self-assembly. The base support may be any ceramic, polymer, metal, silicon, quartz (crystalline silica), glass, zinc oxide, alumina, Paraffin film, polycarbonate (PC), combinations thereof and the like. In some embodiments, the solid support is hollow metal or metal oxide nano- or microspheres.

In some embodiments, the solid support comprises microspheres or beads. As used herein, “microspheres” or “beads” or “particles” or grammatical equivalents herein is meant to include small discrete particles. Suitable bead compositions include, but are not limited to, plastics, ceramics, glass, polystyrene, methyl styrene, acrylic polymers, paramagnetic materials, thoria sol, carbon graphite, titanium dioxide, latex or cross-linked dextrans such as Sepharose, cellulose, nylon, cross-linked micelles and Teflon, as well as any other materials outlined herein for solid supports may all be used. “Microsphere Detection Guide” from Bangs Laboratories, Fishers Ind. is a helpful guide. In certain embodiments, the microspheres are magnetic microspheres or beads. In some embodiments, the beads can be color coded. For example, MicroPlex® Microspheres from Luminex, Austin, Tex. may be used.

The beads need not be spherical; irregular particles may be used. Alternatively, or additionally, the beads may be porous. The bead sizes range from nanometers, i.e. lnm, to millimeters, i.e. 1 mm, with beads from about 0.01 micron to about 200 microns being preferred, and from about 0.5 to about 5 microns being particularly preferred, although in some embodiments smaller or larger beads may be used. In some embodiments, beads can be about 0.1, 0.2, 0.5, 1, 1.5, 2, 2.5, 2.8, 3, 3.5, 4, 4.5, 5, 5.5, 6, 6.5, 7, 7.5, 8, 8.5, 9, 9.5, 10, 10.5, 15, or 20 μm in diameter.

In some embodiments, the solid support has at least one of the components (materials) transparent to electromagnetic radiation. In some embodiments, the transparent substrate is conductive. In some embodiments, the transparent substrate is dielectric. In some embodiments, the solid support has at least one of its components opaque to electromagnetic radiation. In some embodiments, the opaque substrate is conductive. In some embodiments, the opaque substrate is dielectric. In some embodiments, the conductive component (material) is porous. In some embodiments, the dielectric component is porous. In some embodiments, the pore size is less than 10 nm. In some embodiments, one of the components is magnetic. In some embodiments, the substrate has a periodic structure. In some embodiments, the period of the substrate is less than a micron in dimension. In some embodiments, the period of the substrate is more than a micron in dimension. In some embodiments, the substrate has a combination of the above embodiments. In some embodiments, the substrates mentioned above generate an electromagnetic resonance.

In some embodiments, the solid support is a sensor. In some embodiments, the sensor is a piezoelectric sensor. In some embodiments, the piezoelectric sensor has at least a monolayer of gold on top or bottom of the solid support. In some embodiments, the sensors are round-shaped. In some embodiments, solid support comprises quartz crystals with gold coating. In some embodiments, the sensors are mounted on a flowcell.

In some embodiments, the solid support comprises a plurality of materials of defined compositions and property. In some embodiments, the solid support comprises a solid surface with a bilayer of different chemical composition. In some embodiments, one layer may be conductive of electricity. In some embodiments, one layer may be a metal. Exemplary metals include, but are not limited to, silver, gold, copper, platinum, titanium, chromium, Al, Ni, Cr, Fe, ceramic metals such as Indium-tin-oxide etc. or combinations thereof. In some embodiments, one layer comprises magnetic materials, e.g., Fe, Ni, Non-oxide, non-elemental magnetic material Nd2Fe14B, SmCo5 etc., non-elemental, oxide magnetic material Fe₃O₄, Fe₂O₃. In some embodiments, the second layer can be non-conductive or non-magnetic. In some embodiments, the second layer may be ceramic, polymer, metal, silicon, quartz (crystalline silica), glass, zinc oxide, alumina, Paraffin film, polycarbonate (PC), or combinations thereof.

In some embodiments, the solid support is a microarray. In some embodiments, the mircroarray may comprise different materials for the wells and the base. In some embodiments, the solid support is a bead. In some embodiments, the bead may comprise detectable label.

In some embodiments, the bacteria, bacteriophage, and/or the bacteria and bacteriophage complex may be immobilized on a solid support. In some embodiments, bacteria are immobilized on a solid support. In some embodiments, bacteriophage is immobilized on a solid support. In some embodiments, bacteria and bacteriophage complex is immobilized on a solid support. In some embodiments, the bacteria, bacteriophage, or the bacteria and bacteriophage complex are adsorbed passively on the substrate. In some embodiments, at least one bacterium, one bacteriophage, or one bacteria and bacteriophage complex is actively adsorbed by applying an external electric field. In some embodiments, the immobilization is performed under with buffers of varying concentrations and pH.

In some embodiments, the immobilization of the bacteria, bacteriophage, and/or the bacteria and bacteriophage complex to the solid support can be achieved through direct or indirect bonding to the solid support. In some embodiments, the bonding can be by covalent linkage. See, Joos et al. (1997) Analytical Biochemistry, 247:96-101; Oroskar et al. (1996) Clin. Chem., 42:1547-1555; and Khandjian (1986) Mol. Bio. Rep., 11:107-11. In some embodiments, the solid support comprises functional groups capable of immobilizing the bacteria, bacteriophage, or the bacteria and bacteriophage complex.

In some embodiments, the solid supports may comprise functional groups capable of covalently linking the bacteria, bacteriophage, or the bacteria and bacteriophage complex directly or indirectly through chemical linkers. Examples of functional groups include but are not limited to poly L-lysine, aminosilane, epoxysilane, aldehydes, carboxylic groups, azide, alkalyne, maleimide, amino groups, epoxy groups, cyano groups, ethylenic groups, hydroxyl groups, thiol groups.

In some embodiments, the immobilization is achieved through amine functional groups of a polymer, e.g., N-terminus and ε-amino groups of lysine residues, direct amine bonding of a terminal nucleotide of the template or a primer. In some embodiments, the immobilization is achieved through carboxylic acid/ carboxylate functional groups of a polymer, e.g., C-terminus of a protein or a peptide. In some embodiments, the polymer is an aptamer.

In some embodiments, the solid support comprises epoxide functional groups, N-Hydroxysuccinimide (NETS) group. The epoxide functional groups or the NETS group can be used to immobilize the bacteria, bacteriophage, or the bacteria and bacteriophage complex to the solid support.

In some embodiments, the immobilization of the bacteria, bacteriophage, and/or the bacteria and bacteriophage complex to the solid support can be achieved through non-covalent means. In some embodiments, non-covalently immobilizing the bacteria, bacteriophage, and/or the bacteria and bacteriophage complex to the solid support is via a “binding pair,” which refers herein to two molecules which form a complex through a specific interaction. Thus, the bacteria, bacteriophage, and/or the bacteria and bacteriophage complex can be immobilized on the solid support through an interaction between one member of the binding pair linked to the bacteria, bacteriophage, and/or the bacteria and bacteriophage complex and the other member of the binding pair coupled to the solid support. In a preferred embodiment, the binding pair is biotin and avidin, or variants of avidin such as streptavidin or NeutrAvidin™ In some embodiments, the solid support may comprise streptavidin or its variants and bacteria, bacteriophage, and/or the bacteria and bacteriophage complex may comprise biotin. Methods for biotinylating are known in the art (e.g. through primary amine by NHS-PEO12-Biotin, NHS-LC-LC-Biotin, NHS-SS-PEO4-Biotin from Pierce Chemical Co.; through sulfhydryl group by Maleimide-PEO11-Biotin, Biotin-BMCC Sulfhydryl, Iodacetyl-PEO2-Biotin).

In other embodiments, the binding pair consists of a ligand-receptor, a hormone-receptor, an antigen-antibody. Examples of such binding pair include but are not limited to digoxigenin and anti-digoxigenin antibody; 6-(2,4-dinitrophenyl) aminohexanoic acid and anti-dinitrophenyl antibody; 5-Bromo-dUTP (BrdUTP) and anti-BrdUTP antibody; N-acetyl 2-aminofluorene (AAF) and anti-AAF antibody.

The term “compound” in the context of detecting one or more types of cell, bacteria or bacteriophage refers to a molecule or a group of molecules that interacts a nucleic acid or a protein of a bacteriophage or bacteria, or bacterial cell wall or cell membrane. In some embodiments, the compound is permeable to bacterial cell wall and bacterial cell membrane. In some embodiments, the compound binds to dsDNA, ssDNA, dsRNA, or ssRNA. In some embodiments, the compound preferentially binds to dsDNA. In some embodiments, the compound is photoreactive. Non-limiting examples of compound binding to a nucleic acid include propidium monoazide, ethidium monoazide, based dye, propidium iodide, ethidium bromide, phenanthridine dye, acridine dye, indoles, or imidazole dye.

In some embodiments, compound binds covalently to a nucleic acid or a protein. In some embodiments, the compound intercalates between the bases of a nucleic acid. In some embodiments, the compound is modified upon contacting a bacteriophage or bacterial nucleic acid or protein. Non-limiting examples of methods of modification include forming a covalent bond, changing its composition, and changing its electronic structure. In some embodiments, the compound binds to a function group of a protein such as amine, carboxyl, phosphate, aldehyde, thiol, hydroxyl, and carbonyl. In some embodiments, the compound binding to a protein is an antibody. In some embodiments, the compound binding to a protein may be carbodiimide, NHS ester, imidoester, pentafluorophenyl ester, maleimide, haloacetyl (Bromo- or Iodo-), hydrazide, alkoxyamine, diazirine, aryl azide, isocyanate, epoxide, cyanide, and alkyne.

In some embodiments, the compound has a low dissociation constant. In some embodiments, the compound upon binding to a protein or a nucleic acid prevents other type of compounds binding to the same protein or nucleic acid. In some embodiments, a compound preferentially binds to a protein or nucleic acid in the presence of another protein or nucleic acid which is bound to a different compound. In some embodiments, this preferential binding of a compound to one set of nucleic acid or protein in the presence of another set of nucleic acid or protein that is bound to a different compound is at least in part because the different compound prevents binding of the compound to the another set of nucleic acid or protein.

In some embodiments, the compound comprises a detectable label.

The terms “detectable label” and “tag” have been used interchangeably throughout the application and refers to a molecule or a compound or a group of molecules or a group of compounds and being capable of being detected. In some embodiments, detectable label can associate with a molecule and assist in detecting the molecule with which it is associated with.

In some embodiments, detectable label can generate a signal which can be detected. In some embodiments, the detectable label is converted to a different molecule or compound that can generate a signal which can be detected. In some embodiments, the detectable label is part of a binding pair. In some embodiments, the detectable label is associated with a solid support. In some embodiments, the detectable label is associated with a bead. In some embodiments, the detectable is further modified prior to detection. In some embodiments, such modification may include covalent modification. In some embodiments, the detectable label is associated with the bacteriophage, and/or the bacteria prior to the formation of the complex of the bacteriophage and the bacteria. In some embodiments, the detectable label is associated with the bacteriophage, bacteria, and/or the complex of bacteriophage and the bacteria after the formation of the complex.

In some embodiments, the detectable label is associated with a protein or a nucleic acid (e.g., genomic nucleic acid, fragments of genomic nucleic acid, a probe or primer) of bacteria, bacteriophage, or a complex of bacteria and bacteriophage and is used to detect the bacteria, bacteriophage, or a complex of bacteria and bacteriophage. In some embodiments, the detectable label is associated with a protein or a nucleic acid inside the bacteria, bacteriophage, or a complex of bacteria and bacteriophage.

In some embodiments, the bacteriophage, bacteria, and/or the complex of bacteriophage and the bacteria may comprise more than one type of detectable label. In some embodiments, the detectable labels of the bacteriophage, the bacteria, and their complexes are different from each other.

In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluoresence, or chemiluminescence, or any other appropriate means.

Detectable labels include but are not limited to fluorophores, isotopes (e.g., 32P, 33P, 35S, 3H, 14C, 125I, 131I), electron-dense reagents (e.g., gold, silver), nanoparticles, enzymes commonly used in an ELISA (e.g., horseradish peroxidase, beta-galactosidase, luciferase, alkaline phosphatase), chemiluminiscent compound, photoluminescent compounds (e.g., quantum dots, lanthanide particles and lanthanide chelates), colorimetric labels (e.g., colloidal gold), magnetic labels (e.g. Dynabeads™), biotin, digoxigenin, haptens, proteins for which antisera or monoclonal antibodies are available, ligands, hormones, oligonucleotides capable of forming a complex with the corresponding oligonucleotide complement.

In some embodiments, the detectable label comprises a fluorescent moiety. Non-limiting examples of fluorescent moiety include TOTO®, YOYO®, BOBO™ POPO™ SYBR®, SYTOX®, PicoGreen, OliGreen, RiboGreen, Cy2, Cy3, Cy3.5, Cy5, Cy5.5, Cy7, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED™, SPECTRUM GREEN, cyanine dyes (e.g., CY-3™, CY-5™, CY-3.5™, CY-5.5™, etc.), Alexa Fluor® dyes (e.g., Alexa Fluor® 350, Alexa Fluor® 405, Alexa Fluor® 430, Alexa Fluor® 488, Alexa Fluor® 532, Alexa Fluor® 546, Alexa Fluor® 568, Alexa Fluor® 594, Alexa Fluor® 632, Alexa Fluor® 633, Alexa Fluor® 660, Alexa Fluor® 680, etc.), BODIPY™dyes (e.g., BODIPYTM FL, BODIPYTM R6G, BODIPY™ TMR, BODIPY™ TR, BODIPY™ 530/550, BODIPY™ 558/568, BODIPY™ 564/570, BODIPY™ 576/589, BODIPY™ 581/591, BODIPY™630/650, BODIPY™650/665, etc.), IRDyes (e.g., IRD40, IRD 700, IRD 800, etc.), PMA™ (propidium monoazide), propidium iodide, ethidium monoazide (EMA) and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable label may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92: 4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described previously, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/ QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

A suitable detectable label can be an intercalating DNA/RNA dye that have dramatic fluorescent enhancement upon binding to double-stranded DNA/RNA. Examples of suitable dyes include, but are not limited to, SYBRTM and Pico Green (from Molecular Probes, Inc. of Eugene, Oreg.), ethidium bromide, propidium iodide, chromomycin, acridine orange, Hoechst 33258, TOTO-1, YOYO-1, and DAPI (4′,6-diamidino-2-phenylindole hydrochloride). Additional discussion regarding the use of intercalation dyes is provided by Zhu et al., Anal. Chem. 66:1941-1948 (1994), which is incorporated by reference in its entirety.

As used herein, the term “cell” includes both prokaryotic cell and eukaryotic cell. Non-limiting examples of eukaryotic cell includes protozoa cell, yeast cell, plant cell, mammalian cell, human cell. Human cell can be from any human organ, blood, or tissue.

As used herein the term “virus” or “viruses” in the context of cell refers to viruses capable of specifically binding to a specific cell. Examples of such viruses are described in Lodish H, Berk A, Zipursky S L, et al. Molecular Cell Biology. 4th edition. New York: W. H. Freeman; 2000. Section 6.3, Viruses: Structure, Function, and Uses., which is incorporated by reference in its entirety.

As used herein, the term “bacteria” refers to gram positive, gram negative and neither gram positive or gram-negative bacteria. In some embodiments, the bacteria are pathogenic to plants. In some embodiments, the bacteria are pathogenic to crop. Bacterial pathogens for plants are well known in the art. See https://en.wikipedia.org/wiki/Category:Bacterial_plant_pathogens_and_diseases. Non-limiting examples of plant bacterial pathogens include: Candidatus Liberibacter, Candidatus Phytoplasma solani, Clavibacter michiganensis, Pectobacterium atrosepticum, Pectobacterium betavasculorum, Pectobacterium carotovorum, Pectobacterium carotovorum subsp. betavasculorum, Pectobacterium wasabiae, Pseudomonas amygdali, Pseudomonas asplenii, Pseudomonas caricapapayae, Pseudomonas cichorii, Pseudomonas coronafaciens, Pseudomonas corrugata, Pseudomonas ficuserectae, Pseudomonas flavescens, Pseudomonas fuscovaginae, Pseudomonas helianthi, Pseudomonas marginalis, Pseudomonas oryzihabitans, Pseudomonas palleroniana, Pseudomonas salomonii, Pseudomonas savastanoi, Pseudomonas syringae, Pseudomonas tomato, Pseudomonas turbinellae, Pseudomonas viridiflava, Ralstonia solanacearum, Rhodococcus fascians, Xanthomonas campestris, Xanthomonas campestris pv. campestris, Xanthomonas oryzae, and Xylella fastidiosa.

In some embodiments, the bacteria are pathogenic to eukaryotic organisms of the kingdom metazoan. In some embodiments, the bacteria are pathogenic to vertebrates. In some embodiments, the bacteria are pathogenic to mammals. In some embodiments, the bacteria are pathogenic to humans. In some embodiments, the bacteria are pathogenic to farm animals. Non-limiting examples of farm animals include cows, pigs, chicken, sheep, goat, ducks, horse. In some embodiments, the bacteria are pathogenic to domestic animals. In some embodiments, the bacteria are pathogenic to marine animals, insects (bees, butterflies, and mosquitoes).

Non limiting examples of bacteria pathogenic to humans include Citrobacter freundii, Klebsiella pneumoniae, Acinetobacter, Proteus mirabilis, Pseudomonas aeruginosa, Salmonella enteritidi, Escherichia coli, Staphylococcus aureus, Enterococci, Bacillus cereus, Escherichia coli, Listeria monocytogenes, Shigella spp., Staphylococcus aureus, Staphylococcal enteritis, Streptococcus, Vibrio cholerae, (including O1 and non-O1), Vibrio parahaemolyticus, Vibrio vulnificus, Yersinia enterocolitica and Yersinia pseudotuberculosis, Campylobacter jejuni, Clostridium perfringens, Salmonella spp., Salmonella typhimurium, Mycobacterium tuberculosis, Listeria monocytogenes, Shigella spp., Clostridium botulinum, Vibrio vulnificus, Clostridium perfringens, Bacillus cereus, Bacillus anthracis, Campylobacter coli, Yersinia pestis, Bacillus subtilis, Pseudomonas syringae, Dickeya solani, Clavibacter michiganensis, Erwinia carotovora, Agrobacterium tumefaciens, Erwinia amylovora, Enterobacter aerogenes, Enterobacter cloacae, Acinetobacter baumannii, and Clostridium difficile.

In some embodiments, the pathogenic bacteria are food borne. Non-limiting examples of food borne pathogenic bacteria include Bacillus cereus, Escherichia coli, Listeria monocytogenes, Shigella spp., Staphylococcus aureus, Staphylococcal enteritis, Streptococcus, Vibrio cholerae, (including O1 and non-O1), Vibrio parahaemolyticus, Vibrio vulnificus, Yersinia enterocolitica and Yersinia pseudotuberculosis, Campylobacter jejuni, Clostridium perfringens, Salmonella spp., Salmonella typhimurium.

Non-limiting examples of pathogenic bacteria and their corresponding bacteriophages are shown in Table 1 below.

TABLE 1
Pathogenic bacteria, corresponding bacteriophages and the associated diseases

| Bacteria | Bacteriophage | Disease |
|---|---|---|
| Mycobacterium | Mycobacteriophage D29, TM4, phAE159, Che12 | Tuberculosis |
| Tuberculosis | (and their derivatives) | |
| Bacillus | γ-phage, Wβ | Anthrax |
| anthracis | | |
| Campylobacter | NCTC 12673 | Gastroenteritis, acute |
| jejuni | | enterocolitis |
| Campylobacter | GP047 | Gastroenteritis, acute |
| coli | | enterocolitis |
| E. coli | CBA120, AR1 and bacteriophage 56 | Gastroenteritis, |
| | | urinary tract |
| | | infections, and |
| | | neonatal meningitis |
| E. coli O157 | LG1 | |
| Yersinia pestis | ΦA1122, ΦA1122, H, P, Y, Tal, 513, L-413C | Plague |
| Pseudomonas | PA5oct, NCIMB 101116, KT28, vB_PaeM_MAG1 | Pneumonia, Septic |
| aeruginosa | (MAG1) and vB_PaeP_MAG4 (MAG4) | shock, Urinary tract |
| | | infection, |
| | | Gastrointestinal |
| | | infection, skin and soft |
| | | tissue infections |
| Bacillus | SPO2, Φ105, β22 | |
| subtilis | | |
| Pseudomonas | phi 6, φPsa17 | Bacterial speck in |
| syringae | | tomato |
| Dickeya solani | vB_DsoM_LIMEstone1 and vB_DsoM_LIMEstone2 | Wilts and stem rots |
| (Dickeya spp.) | | |
| Clavibacter | CMP1, CN77 | Systemic vascular |
| michiganensis | | infection |
| Erwinia | T4, ZF40, phage 59 | Plant cell wall |
| carotovora | | destruction |
| Agrobacterium | PB2, PB21, PS8, R4 | Crown gall disease |
| tumefaciens | | |
| Erwinia | phiEa2809, vB_EamM_Ea35-70 (Ea35-70), Y2 | Fireblight |
| amylovora | | |
| Enterobacter | F20 | Sepsis |
| aerogenes | | |
| Citrobacter | phi I, phi II, and phi III | Nosocomial infections |
| freundii | | of the respiratory tract, |
| | | urinary tract, blood |
| Enterobacter | Ent, 1 | Urinary tract and |
| cloacae | | respiratory tract |
| | | infections |
| Shigella spp. | Sh, φSboM-AG3, EP23, SP18, Stix | Shigellosis |
| Klebsiella | 0507-KN2-1, KP34, JD001, KI3 | Nosocomial |
| pneumoniae | | infections. |
| Acinetobacter | vB_AbaM-IME-AB2, ZZ1, AB1, AP22, and phiAC-1 | Nosocomial |
| baumannii | | infections. |
| Staphylococcus | MR-5, Ph10, Ph12, U14, H96, JS01, 88, | skin infections, such |
| aureus | YMC/09/04/R1988 MRSA BP | as pimples, impetigo, |
| | | boils, cellulitis, |
| | | folliculitis, carbuncles, |
| | | scalded skin |
| | | syndrome, and |
| | | abscesses, pneumonia, |
| | | meningitis, |
| | | osteomyelitis, |
| | | endocarditis, toxic |
| | | shock syndrome, |
| | | bacteremia, and sepsis |
| Clostridium | phiC2, phiC5, phiC6, phiC8, PhiCD119, phiCD6356, | Diarrhea, Colitis, |
| difficile | phiCD38-2 | Sepsis |
| Listeria | A511, A513, A507, A502, A505, A519, A528, A020, | Listeriosis |
| | B024, B012, B035, C707, A118, P100, PSA, A006, | |
| | A118, A500, B025, P35, P40, 20422-1, 805405-1 | |
| Salmonella | Felix 01, SPN2T, SPN3C, SPN8T, SPN9T, SPN11T, | Diarrhea, fever, and |
| | SPN13B, SPN16C, SPN4S, SPN5T, SPN6T, SPN7C, | abdominal cramps |
| | SPN9C, SPN14, SPN18, SPN1S, SPN2TCW, | |
| | UAB_Phi20, UAB_Phi78, and UAB_Phi87 | |
| Xanthomonas | Cp1, Cp2, Cp1-sensitive, XacF1, Phil7 | Citrus canker |
| axonopodis | | |
| Brucella | Berkeley, Tbilisi, Firenze (Fz), Weybridge (Wb), | Brucellosis |
| | S708, R/C, 1066, 281, 02, 177, 110, V, 11sa, 544, 141 | |
| Helicobacter | KHP30, KHP40, HP1 | Infection of the |
| pylori | | stomach, peptic ulcer |
| | | and stomach cancer |
| Leptospira | LE1 | Leptospirosis |
| Chlamydia | Chp1, Chp2, Chp3, φCPG1 φCPAR39 (φCpn1), | Psittacosis or |
| | Chp4, φAR39 | respiratory tract |
| | | diseases, enteritis and |
| | | chronic bowel |
| | | diseases, ornithosis, |
| | | pneumonia |
| Legionella | Mu | Legionnaires disease |
| pneumophila | | |

In some embodiments, the bacteria are non-pathogenic to mammals. In some embodiments, the bacteria are non-pathogenic to humans. In some embodiments, the bacteria are mesophilic. In some embodiments, the bacteria are thermophilic.

In some embodiments, the bacteria are used in the fermentation industry. In some embodiments, the bacteria are used in the dairy industry. In some embodiments, the bacteria are used for the fermentation of milk. In some embodiments, the bacteria are used for the fermentation of milk to produce cheese. Non-limiting examples of bacteria used in the dairy industry include: Lactococcus lactis ssp. cremoris; Lactococcus lactis ssp. lactis biovar diacetylactis; Lactococcus lactis ssp. lactis; Leuconostoc mesenteroides ssp. cremoris; Lactobacillus acidophilus; Lactobacillus delbrueckii ssp. bulgaricus; Lactobacillus delbrueckii ssp. lactis; Lactobacillus helveticus; Streptococcus thermophilus; Bifidobacterium bifidus; Brevibacterium linens coryneform bacteria; and Propionibacterium freudenreichii ssp. shermanii.

In some embodiments, the bacteria are used is the wine industry for the malolactic acid fermentation to convert malic acid to lactic acid. Non-limiting examples of bacteria used in the wine industry include lactic acid bacteria of the genera: Lactobacillus, Leuconostoc, Pediococcus, Lactococcus, and Streptococcus, Bifidobacterium, Aerococcus, Carnobacterium, Enterococcus, Oenococcus, Sporolactobacillus, Tetragenococcus, Vagococcus, and Weissella; and Oenococcus oeni.

Non-limiting examples of bacteria used for fermentation in dairy and wine industry and their corresponding bacteriophages are shown in Table 2 below.

TABLE 2
bacteria used for fermentation in dairy and wine industry and their
corresponding bacteriophages

| Bacteria | Phage |
|---|---|
| Lactobacillus casei | A2 |
| Lactobacillus paracasei | CL1, CL2 |
| Lactobacillus paracasei (ssp. paracasei) | PL-1 |
| Lactobacillus plantarum | B1, B2, PhiJL-1, fri, Φg1e, |
| | ΦLP65, ΦJL1 |
| Lactococcus lactis | P001, 949, c6A, P087, |
| | P107, BK5-T, c2, p2, sk1, |
| | ul36, bIL170, bIL67, Q54, |
| | 1706, P335, Tuc2009, r1t |
| Lactococcus lactis (ssp. cremoris) | 1358, 936, KSY1 |
| Lactococcus lactis (ssp. diacetylactis) | P008, P270, P369 |
| Lactococcus lactis (ssp. lactis) | 1483 |
| Lactobacillus casei | A2, ΦAT3, ΦA2, |
| Lactobacillus paracasei | CL1, CL2 |
| Lactobacillus paracasei (ssp. paracasei) | PL-1 |
| Oenococcus oeni | Lco22 |
| Streptococcus group C | a/C7 |
| Streptococcus mitis | |
| Streptococcus mutans | M102, M102AD |
| Streptococcus pneumoniae | Cp-1 (SOCP), Dp-1 |
| Streptococcus thermophilus | Ba 24, DT1, Q1, 2972, |
| | 858, ALQ13.2, ΦAbc2, |
| Thermus thermophilus | ψYS40 |
| Leuconostoc mesenteroides | pro2 |
| Leuconostoc mesenteroides (ssp. cremoris) | Φ400 |
| Lactobacillus delbrueckii | LdlS, Ld3, Ld17, Ld25A, |
| | lb539, LL-Ku, c5, |
| | JCL1032, ΦLL-H, Φmv4, |
| Lactobacillus helveticus | ΦAQ113 |
| Lactobacillus gasseri | KC5a |
| Lactobacillus johnsonii | Lj771 |
| Lactobacillus gasseri | Φadh, ΦKC5a |
| Lactobacillus rhamnosus | ΦLc-Nu |
| Lactobacillus fermentum | EcoSau, EcoInf |

In some embodiments, the bacteria are used in meat fermentation, vegetable fermentation, food fermentation. Exemplary list of bacteria used in meat fermentation includes, but is not limited to Lactobacillus curvatus, Lactobacillus sake, Pediococcus acidilactici, Pediococcus pentosaceus, Lactobacillus plantarum, Micrococcus varians, Staphylococcus carnosus, and Staphylococcus xylosus.

In some embodiments, the bacteria are lactic acid bacteria (LAB) used in the conversion of lactose to lactic acid. In some embodiments, the LAB belongs to the genera Aerococcus, Alloiococcus, Atopobium, Bifidobacterium, Carnobacterium, Enterococcus, Lactobacillus (Lb.), Lactococcus (L.), Leuconostoc (Leuc.), Oenococcus, Pediococcus, Streptococcus (S.), Tetragenococcus, Vagococcus and Weissella (W.)

As used herein the term “susceptible to the bacteriophage” refers to bacteria that have receptors for the bacteriophage that are present in a sample. As a result, the bacteriophages present in a sample can specifically bind to such receptors and infect the bacteria.

As used herein the term “bactericide” or “bactericide” sometimes abbreviated Bcidal, is a substance that kills bacteria. Bactericides are disinfectants, antiseptics, lysozyme, or antibiotics.

Non-limiting examples of disinfectants include active chlorine (i.e., hypochlorites, chloramines, dichloroisocyanurate and trichloroisocyanurate, wet chlorine, chlorine dioxide, etc.), active oxygen (peroxides, such as peracetic acid, potassium persulfate, sodium perborate, sodium percarbonate, and urea perhydrate), iodine (povidone-iodine, Lugol's solution, iodine tincture, iodinated nonionic surfactants), concentrated alcohols (mainly ethanol, 1-propanol, called also n-propanol and 2-propanol, called isopropanol and mixtures thereof; further, 2-phenoxyethanol and 1- and 2-phenoxypropanols are used), phenolic substances (such as phenol (also called “carbolic acid”), cresols such as thymol, halogenated (chlorinated, brominated) phenols, such as hexachlorophene, triclosan, trichlorophenol, tribromophenol, pentachlorophenol, salts and isomers thereof), cationic surfactants, such as some quaternary ammonium cations (such as benzalkonium chloride, cetyl trimethylammonium bromide or chloride, didecyldimethylammonium chloride, cetylpyridinium chloride, benzethonium chloride) and others, non-quaternary compounds, such as chlorhexidine, glucoprotamine, octenidine dihydrochloride etc.), strong oxidizers, such as ozone and permanganate solutions; heavy metals and their salts, such as colloidal silver, silver nitrate, mercury chloride, phenylmercury salts, copper sulfate, copper oxide-chloride etc.; strong acids (phosphoric, nitric, sulfuric, amidosulfuric, toluenesulfonic acids), pH<1; and alkalis (sodium, potassium, calcium hydroxides), such as of pH>13, particularly under elevated temperature (above 60 ° C.), kills bacteria.

Non-limiting examples of antiseptics include properly diluted chlorine preparations (e.g., Dakin's solution, 0.5% sodium or potassium hypochlorite solution, pH-adjusted to pH 7-8, or 0.5-1% solution of sodium benzenesulfochloramide (chloramine B)); some iodine preparations, such as iodopovidone in various galenics (ointment, solutions, wound plasters), in the past also Lugol's solution; peroxides such as urea perhydrate solutions and pH-buffered 0.1-0.25% peracetic acid solutions; alcohols with or without antiseptic additives, used mainly for skin antisepsis; weak organic acids such as sorbic acid, benzoic acid, lactic acid and salicylic acid; some phenolic compounds, such as hexachlorophene, triclosan and Dibromol; and cationic surfactants, such as 0.05-0.5% benzalkonium, 0.5-4% chlorhexidine, 0.1-2% octenidine solutions.

Non-limiting examples of antibiotics include beta-lactam antibiotics (penicillin derivatives (penams), cephalosporins (cephems), monobactams, and carbapenems) and vancomycin, daptomycin, fluoroquinolones, metronidazole, nitrofurantoin, co-trimoxazole, telithromycin, aminoglycosidic antibiotics.

As used herein the term “bacteriostatic agent” refers to a biological or chemical agent that stops bacteria from reproducing, while not necessarily killing them otherwise. Depending on their application, bacteriostatic antibiotics, disinfectants, antiseptics and preservatives can be distinguished. When bacteriostatic antimicrobials are used, the duration of therapy must be sufficient to allow host defense mechanisms to eradicate the bacteria. Upon removal of the bacteriostatic agent, the bacteria usually start to grow again. This is in contrast to bactericides, which kill bacteria.

Non-limiting examples of bacteriostatic antibiotic include Chloramphenicol, Clindamycin, Ethambutol, Lincosamides, Macrolides, Nitrofurantoin, Novobiocin, Oxazolidinone, Spectinomycin, Sulfonamides, Tetracyclines, Tigecycline, Trimethoprim.

As used herein the term “cell-free bacterial receptors” means bacterial receptors for bacteriophages that are substantially free of intact bacterial cells. Substantially free of intact bacterial cells means bacterial receptors that are at least 90%, 95%, 96%, 97%, 98%, 99% or more free of intact bacterial cells. In some embodiments, the bacterial receptors are part of bacterial cell wall fragments or bacterial cell membrane fragments. In some embodiments, the bacterial receptors are isolated from intact bacteria. In some embodiments, the bacterial receptors are purified. In some embodiments, the bacterial receptors are recombinant proteins.

Several amino acid sequences of bacteriophage receptor of bacteria are available in the NCBI protein database. Non-limiting examples of bacteriophage receptor of bacteria include NCBI protein database accession numbers: BAA35202.1, BAA35203, CDH64094.1, CCJ43022.1, EMD14689.1, EKU06323.1, EKU00687.1, AM074452.1, APC75070.1, BAT57805.1, CBJ83363.1, EMV90632.1, ADR59974.1 (https://www.ncbi.nlm.nih.gov/protein).

As used herein, the term “bacteriophage” refers to a virus that infects and can replicate within a bacterium. Bacteriophages may have a lytic cycle, a lysogenic cycle, or both. Bacteriophages attach to specific receptors on the surface of bacteria, including lipopolysaccharides, polyglycans, teichoic acids, proteins, or even flagella to form a bacteria and bacteriophage complex. This specificity means a bacteriophage can infect only certain bacteria bearing receptors to which they can bind, which in turn determines the phage's host range. Host growth conditions also influence the ability of the phage to attach and invade them.

In some embodiments, the receptor binding element of a bacteriophage binds specifically to the receptors of bacteria. In some embodiments, the receptor binding elements are receptor binding proteins of a bacteriophage.

Several bacteriophage nucleic acid sequences of bacterial receptor binding protein are available in the protein database of National Center for Biotechnology Information (NCBI). Non-limiting examples include Enterobacteria phage T4 tail fiber protein gene, tail fiber protein 36 and tail fiber protein 37 genes (GenBank Accession No: J02509), Host interaction protein of bacteriophage J1 (GenBank Accession No. KC171646.1). Each of which is incorporated by reference in the entirety.

Several bacteriophage amino acid sequences of bacterial receptor binding protein are available in the protein database of National Center for Biotechnology Information (NCBI). Non-limiting examples include Chain A, Structure Of The Receptor-binding Protein Of Bacteriophage Det7: A Podoviral Tailspike In A Myovirus (Accession No. 2V5I_A), Chain C, Structure Of Lactococcal Bacteriophage P2 Receptor Binding Protein In Complex With A Llama Vhh Domain (Accession No. BSE_C), Chain B, Structure Of Lactococcal Bacteriophage P2 Receptor Binding Protein In Complex With A Llama Vhh Domain (Accession No. 2BSE_B), Chain C, Structure Of Lactococcal Bacteriophage P2 Receptor Binding Protein (Accession No. 2BSD_C), Chain B, Structure Of Lactococcal Bacteriophage P2 Receptor Binding Protein (Accession No. 2BSD_B), Chain A, Structure Of Lactococcal Bacteriophage P2 Receptor Binding Protein (Accession No. 2BSD_A), Chain A, Structure Of The Receptor-Binding Domain Of The Bacteriophage T4 Short Tail Fibre (1OCY_A), Chain C, Structure Of The Bacteriophage T4 Long Tail Fibre Needle-Shaped Receptor-binding Tip (2XGF_C), Bacteriophage T7 tail fiber protein 37 (GenBank Accession No. AAA32514.1), Bacteriophage J1 Host interaction protein (Gen Bank Accession No. AGZ17304.1). Each of which is incorporated by reference in the entirety.

Genomes of the bacteriophages can be RNA or DNA. In some embodiments, the genome of the bacteriophage can be linear dsDNA, circular dsDNA, circular ssDNA, segmented dsRNA, linear ssRNA. In some embodiments, the phage nucleic acid is nicked. The classification by the International Committee on Taxonomy of Viruses (ICTV) according to morphology and nucleic acid is shown in Table 3.

TABLE 3
ICTV classification of prokaryotic (bacterial and archaeal) viruses

| | | | Nucleic | |
|---|---|---|---|---|
| Order | Family | Morphology | acid | Examples |
| Caudovirales | Myoviridae | Nonenveloped, | Linear | T4 phage, Mu, PBSX, |
| | | contractile tail | dsDNA | P1Puna-like, P2, I3, |
| | | | | Bcep 1, Bcep 43, Bcep |
| | | | | 78 |
| Caudovirales | Siphoviridae | Nonenveloped, | Linear | λ phage, T5 phage, phi, |
| | | noncontractile tail | dsDNA | C2, L5, HK97, N15 |
| | | (long) | | |
| Caudovirales | Podoviridae | Nonenveloped, | Linear | T7 phage, T3 |
| | | noncontractile tail | dsDNA | phage, Φ29, P22, P37 |
| | | (short) | | |
| Ligamenvirales | Lipothrixviridae | Enveloped, rod- | Linear | Acidianus filamentous |
| | | shaped | dsDNA | virus 1 |
| Ligamenvirales | Rudiviridae | Nonenveloped, rod- | Linear | Sulfolobus islandicus |
| | | shaped | dsDNA | rod-shaped virus 1 |
| Unassigned | Ampullaviridae | Enveloped, bottle- | Linear | |
| | | shaped | dsDNA | |
| Unassigned | Bicaudaviridae | Nonenveloped, | Circular | |
| | | lemon-shaped | dsDNA | |
| Unassigned | Clavaviridae | Nonenveloped, rod- | Circular | |
| | | shaped | dsDNA | |
| Unassigned | Corticoviridae | Nonenveloped, | Circular | |
| | | isometric | dsDNA | |
| Unassigned | Cystoviridae | Enveloped, | Segmented | |
| | | spherical | dsRNA | |
| Unassigned | Fuselloviridae | Nonenveloped, | Circular | |
| | | lemon-shaped | dsDNA | |
| Unassigned | Globuloviridae | Enveloped, | Linear | |
| | | isometric | dsDNA | |
| Unassigned | Guttaviridae | Nonenveloped, | Circular | |
| | | ovoid | dsDNA | |
| Unassigned | Inoviridae | Nonenveloped, | Circular | M13 |
| | | filamentous | ssDNA | |
| Unassigned | Leviviridae | Nonenveloped, | Linear | MS2, Qβ |
| | | isometric | ssRNA | |
| Unassigned | Microviridae | Nonenveloped, | Circular | ΦX174 |
| | | isometric | ssDNA | |
| Unassigned | Plasmaviridae | Enveloped, | Circular | |
| | | pleomorphic | dsDNA | |
| Unassigned | Tectiviridae | Nonenveloped, | Linear | |
| | | isometric | dsDNA | |

In some embodiments, the bacteriophages are lytic phages. In some embodiments, the bacteriophages belong to Siphoviridae and Podoviridae families. In some embodiments, the bacteriophages are responsible for milk fermentation failures. In some embodiments, the bacteriophages are responsible for infecting the bacteria useful for bacterial fermentation of milk. In some embodiments, the bacteriophages are responsible for infecting the bacteria useful for malolactic fermentation in wine industry.

As used herein the phrase “complex of bacteriophage and bacteria” or the “complex” refers to a state in the bacteriophage infection in which the bacteriophages attach to specific receptors on the surface of bacteria, including lipopolysaccharides, glycoprotein, polyglycans, teichoic acids, proteins, or even flagella to form a bacteria and bacteriophage complex. The complex includes stages prior to, during, or after introduction of bacteriophage genetic material into the bacteria while the bacteriophage remains adhered to the bacterial surface.

In some embodiments, the complex is separated from the incubation mixture comprising bacteriophage and bacteria prior to detection of the complex. In some embodiments, the separation is by centrifugation, filtration, application of electric field, size exclusion chromatography or a combination thereof

In some embodiments, the complex is detectably labeled after the formation of the complex. In some embodiments, the bacteriophage and/or the bacteria are detectably labeled resulting in a detectably labeled complex. In some embodiments, the detectable labels for the bacteriophage and the bacteria are different. In some embodiments, the detectable labels for the bacteriophage, the bacteria, and/or the complex are different. In some embodiments, the complex can comprise more than one type of detectable label.

“Sample” can be from a human or a non-human origin. A sample may include a specimen of natural or synthetic origin. In some embodiments, sample can be a biological sample such as tissues, tissue homogenate, feces, bodily fluids, inoculums, and cheeck swab. “Bodily fluids” may include, but are not limited to, blood, serum, plasma, saliva, cerebral spinal fluid, tears, lactal duct fluid, lymph, sputum, mucus, pleural fluid, urine, amniotic fluid, and semen. A sample may include a bodily fluid that is “acellular.” An “acellular bodily fluid” includes less than about 1% (w/w) whole cellular material. Plasma or serum are examples of acellular bodily fluids.

In some embodiments, the sample can be fermentation samples. Non-limiting examples of fermentation samples include bacterial fermentation samples, dairy fermentation samples, yeast fermentation samples, starter culture, milk starter culture, whey starter culture. In some embodiments, the sample can be water, air, aerosol, liquid collected from facility equipment, swabbed facility surfaces, fluid samples from facilities, wash water from a facility, wash water of produce, swabbed ground samples, swabbed food sample, fermented liquid, bacterial culture, bacterial broth, sewer, soil.

In some embodiments, the sample can be food. Food can be processed, unprocessed, cooked, or raw. Non-limiting examples of food sample include raw milk, pasteurized milk, skim milk, whey, cheese, fruits, vegetables, meat, shrimp, crab, clams, fish, grains, nuts. In some embodiments, the sample can be drinks and beverages. Non-limiting examples include coconut milk, soy milk, tea, coffee, lemonade, fruit juice, vegetable juice, and soda.

In some embodiments, sample can be from a patient suspected of having a bacterial infection. Patients can be human and non-human animals. In some embodiments, sample can be from non-human animals. In some embodiments, sample can be from farm animals, marine animals, domestic animals, wild animals, insects.

In some embodiments, sample can be from plants. Non-limiting examples of plant sample include leaves, roots, stem, nuts, grains, plant extract, root extract, petals, fruit, bark, seed.

As used herein the term “device” refers to a widget that aids in detecting or can detect bacteria, bacteriophages or bacteriophage and bacteria complex in a sample.

In some embodiments, the device comprises a planar surface. In some embodiments, the planar surface comprises a bilayer of two different compositions. In some embodiments, one layer may be conductive of electricity. In some embodiments, one layer may be a metal. Exemplary metals include, but are not limited to silver, gold, copper, platinum, titanium, chromium, Al, Ni, Cr, Fe, ceramic metals such as Indium-tin-oxide etc. or combinations thereof In some embodiments, one layer comprises magnetic materials, e.g., Fe, Ni, Non-oxide, non-elemental magnetic material Nd₂Fe₁₄B, SmCo₅ etc., non-elemental, oxide magnetic material Fe₃O₄, Fe₂O₃. In some embodiments, the second layer can be non-conductive or non-magnetic. In some embodiments, the second layer may be ceramic, polymer, metal, silicon, quartz (crystalline silica), glass, zinc oxide, alumina, Paraffin film, polycarbonate (PC), or combinations thereof In some embodiments, the planar surface is in fluid communication with the sample comprising bacteriophage, bacteria, and/or a complex of bacteriophage and bacteria.

In some embodiments, the device comprises solid support. In some embodiments, the solid supports may comprise functional groups capable of covalently linking the bacteriophage, bacteria, or the complex of bacteriophage and bacteria directly or indirectly through chemical linkers. Examples of functional groups include but are not limited to poly L-lysine, aminosilane, epoxysilane, aldehydes, amino groups, epoxy groups, cyano groups, ethylenic groups, hydroxyl groups, thiol groups, epoxide N-Hydroxysuccinimide (NHS) group. In some embodiments, the solid support may comprise one member of a binding pair and can immobilize a bacteriophage, bacteria, or the complex of bacteriophage and bacteria comprising the other member of the binding pair.

In some embodiments, the device may comprise a sensor. In some embodiments, the device may comprise plurality of sensors. In some embodiments, the sensor is a photo detector. In some embodiments, the photo detector is a spectrometer. In some embodiments, the sensor is a Raman spectrometer. In some embodiments, the sensor can detect surface plasmon resonance. In some embodiments, the sensor can detect change in mass. In some embodiments, the sensor can detect change in resonance frequency of the sensor. In some embodiments, the sensor comprises a crystal and the sensor can detect dissipation of shear movement of the crystal. In some embodiments, the sensor comprises piezoelectric crystal. In some embodiments, the sensor comprises piezoelectric crystal and a monolayer of a metal such as gold, silver etc. In some embodiments, the device comprises a sensor and a flow cell. In some embodiments, sample is flown over the sensor by a continuous flow.

In some embodiments, the device is fluidic device. In some embodiments, the fluidic device may comprise a solid support. In some embodiments, fluid is introduced to the solid support. The fluid may be stationary or the fluid may have a relative flow with respect to the solid support. In some embodiments the fluid may comprise bacteriophage, bacteria, and/or complexes of bacteriophage and bacteria. In some embodiments, the fluidic device may have two solid supports opposite to each other. In some embodiments, the fluidic device is a fluidic chamber. In some embodiments, bacteriophage, bacteria, and/or complexes of bacteriophage and bacteria are in contact with the solid support.

In some embodiments, the device comprises of at least one fluidic chamber or channel comprising at least two electrodes and at least one fluidic connection between the electrodes. In some embodiments, the device measures the membrane potential in which a change in the membrane potential is indicative of phage infection of a bacterial cell. In some embodiments, the device measures the redox potential of a molecule. In some embodiments, voltage sensitive dyes are used to measure response of the bacterial cells to phage infection. In some embodiments, application of an electric field between the electrodes allows the differential migration of the bacteriophage, bacteria, or the complex of the bacteriophage and the bacteria to the positive electrode. In some embodiments, the device may comprise a sensor to detect the differential migration of the bacteriophage, bacteria, or the complex of the bacteriophage and the bacteria. In some embodiments, a direct current is applied. In some embodiments, an alternate current is applied.

In some embodiments, the fluidic device comprises at least one main channel adapted to carry the fluid. In some embodiments, the channel comprises an interior wall. In some embodiments, the fluidic device comprises an inlet module upstream of the main channel and/or an outlet module downstream of the main channel. In some embodiments, the solid support comprises a hydrophobic surface. In some embodiments, the fluidic device comprises a force transducer that controls flow of the fluid. In some embodiments, the force transducer is an electric field, a magnetic field, a mechanical force, an optically induced force or any combination thereof

In some embodiments, the device comprises a surface plasmon resonance (SPR) system. In some embodiments, the SPR system comprises a source of polarized light, a sensor comprising a transparent element such as a prism covered with a thin metal film and linking layer, a flow chamber, a system for controlling transport of fluid, and an optical unit for detecting reflected light. The SPR principle is based on the excitation of surface plasmons in a thin layer of a metal such as gold or silver, using polarized light. Polarized light from the prism is reflected from the metal surface, and at a certain angle of light incidence the excitation of resonance in the metal film results in intensity and phase changes in the reflected light beam. An evanescent field is generated which travels in a direction perpendicular to the surface, i.e. in the direction of the fluid sample. As a result of excitation of surface plasmons the incident light is adsorbed, resulting in a decrease in the intensity of the reflected light. For each wavelength and the corresponding effective refractive index ratios between both sides of the metal layer, there is a specific angle at which a minimum in reflectivity is observed, the SPR angle. This angle increases with decreasing wavelength. When interactions occur on the metal surface within the range of the penetration depth of the evanescent field while at the glass phase the refractive index remains constant, a change in refractive index of the dielectric fluid sample occurs. This change in refractive index causes a change in the angle at which SPR occurs. This change in SPR angle is recorded as a shift in the SPR angle with time, resulting in SPR sensorgrams. The change in SPR angle of reflected light is directly proportional to the binding of the bacteriophage, bacteria, and/or the complex of bacteriophage and bacteria on the metal surface of the prism.

In some embodiments, the device comprises a calorimeter. The device measures the heat of chemical reactions or physical changes, endothermic and exothermic peaks, enthalpy as well as heat capacity.

Detection

In some embodiments, the bacteriophage, bacteria, and/or the complex is excited by a specific electromagnetic radiation (excitation) with a defined energy and amplitude and the complex is detected by a specific electromagnetic spectral signature that is generated by the complex in response to the exciting electromagnetic radiation. In some embodiments, the complex is detected by spectrometer, optical microscope, Raman spectroscopy, surface enhanced Raman spectroscopy, SPR.

In some embodiments, the bacteriophages are detected by Raman spectroscopy or surface enhanced Raman spectroscopy (SERS). Samples comprising the bacteriophage, bacteria, and the complex are introduced to a solid support comprising a metal surface. In some embodiments, the metal surface comprises nanostructures. In some embodiments, the metal surface comprises nanoparticles arranged on the solid support by self-assembly. In some embodiments, the metal surface comprises functional groups for immobilizing the bacteriophage, bacteria, and/or the complex. In some embodiments, optical excitation of the samples can be carried out at 532 nm. The corresponding Raman shift from the sample can be measured by detecting the emission from the sample by a spectrometer. In some embodiments, Raman spectroscopy or SERS can be carried out in aqueous environment.

In some embodiments, the bacteria or the bacteriophage are detected by SPR. In some embodiments, the bacteriophage or the bacteria are immobilized on flow cells of a sensor surface. When a sample comprising the other partner (i.e. bacteriophage or the bacteria) which is not immobilized and free in solution, passes through the flow cell, the resulting complex is immobilized on the flow cell and the resulting signal is detected. In some embodiments, the sample flows on the flow cell at a constant rate.

In some embodiments, the bacteria or the bacteriophage are detected by electron microscopy. In some embodiments, the solid support comprises silicon nitride. In some embodiments, the solid support comprises functional groups to immobilize the bacteriophage and/or the bacteria. In some embodiments, the sample comprising bacteriophage and/or bacteria are introduced to the solid support by a flow cell. In some embodiments, a contrast reagent is added to the sample comprising bacteriophage and/or bacteria. Non-limiting examples of contrast reagent include uranyl formate, sodium phospho-tungstate, ammonium molybdate, methyl amine tungstate. In some embodiments, the transmission electron images are recorded on a CCD camera with magnification greater than 1000×.

In some embodiments, the bacteria or the bacteriophage are detected by scanning probe microscopy. In some embodiments, the detection is by atomic force microscopy. In some embodiments, the sample comprising bacteriophage, bacteria and/or the complex are introduced to the solid support. In some embodiments the bacteriophage, bacteria and/or the complex are immobilized on a solid support. In some embodiments, the solid support is glass. In some embodiments, the solid support may further comprise metal.

In some embodiments, the scanning probe is operated in constant height mode, deflection mode, tapping mode, or lift mode. In some embodiments, the morphology of the sample is recorded as height, amplitude or deflection of the probe. In some embodiments, the phage infection is deduced from the morphology data by comparing it with uninfected sample. In some embodiments, the scanning probe tip is biased by applying an alternate voltage or a direct current voltage. In some embodiments, a map of the contact potential of the phage infected bacteria is compared to the uninfected bacteria to evaluate susceptibility. In some embodiments, the probe tip comprises a thermal sensor. In some embodiments, the thermal sensor is a metal wire. In some embodiments, the probe tip forms one leg of a Wheatstone bridge. In some embodiments, the bridge is used to maintain the scanning probe tip at to measure the temperature of the scanning tip probe, or to maintain the tip at a constant temperature. In some embodiments, the heat flow from the sample is measured by at least one tip. In some embodiments, the heat flow from the sample measured by scanning at least one tip over the sample in the temperature contrast mode by measuring the resistance change of the probe. In some embodiments, the heat flow from the sample is measured in the current contrast mode by supplying additional energy to the circuit. In some embodiments, the energy required to maintain the constant current within the tip is a measured as the heat flow from the sample. In some embodiments, the difference in the heat flow from the sample to that of a negative control that does not contain the infectious phages is used to determine the infectious phages in the sample.

In some embodiments, the bacteria are detected by electrochemical device. In some embodiments, the device comprises of at least one fluidic chamber or channel comprising at least two electrodes and at least one fluidic connection between the electrodes. In some embodiments, the device comprises one or more sensors. In some embodiments, the bacteriophage, bacteria, and/or the complex are immobilized on a solid support of the device. In some embodiments, sample comprising the bacteriophage, bacteria, and/or the complex are flowed at a constant rate over the sensor of the device.

In some embodiments, the electrical current is measured as a function of the applied voltage to the electrodes. In some embodiments, the device measures the membrane potential in which a change in the membrane potential is indicative of phage infection of a bacterial cell. In some embodiments, the device measures the redox potential of a molecule. In some embodiments, the pH change is measured. In some embodiments, the enzyme activity is measured (NAD-NADH, ADP-ATP). In some embodiments, voltage sensitive dyes are used to measure response of the bacterial cells to phage infection. In some embodiments, the fluorescence spectrum of a dye is monitored as a function of membrane potential change.

In some embodiments, application of an electric field between the electrodes allows the differential migration of the bacteriophage, bacteria, or the complex of the bacteriophage and the bacteria to the positive electrode. In some embodiments, the device may comprise a sensor to detect the differential migration of the bacteriophage, bacteria, or the complex of the bacteriophage and the bacteria. In some embodiments, a direct current is applied. In some embodiments, an alternate current is applied.

In some embodiments, the bacteria or the bacteriophages are detected by change in mass. In some embodiments, the bacteriophage and/or the bacteria is immobilized on a solid support. In some embodiments, the immobilization is by a covalent bond between the functional groups and the bacteriophage, bacteria, and/or the complex. In some embodiments, the immobilization is by a non-covalent means, e.g., binding of a binding pair, e.g. biotin/streptavidin.

In some embodiments, the solid support comprises a sensor. In some embodiments, the sample comprising the bacteriophage, bacteria, and/or the complex is flowed over the sensor. In some embodiments, the bacteria or the bacteriophages are measured as a change in the mass upon phage complex formation. In some embodiments, the change in the mass on the sensor is detected by measuring the change is the resonance frequency of the sensor. In some embodiments, the sensor comprises a crystal. In some embodiments, the change is the mass of the sensor is derived by measuring the dissipation of shear movement of the crystal.

In some embodiments, the sensor is a piezoelectric sensor. In some embodiments, the piezoelectric sensor has at least a monolayer of metal, e.g., gold, silver, platinum etc. on top or bottom of the sensor surface. In some embodiments, the sensors are round-shaped. In some embodiments, the sensor is a quartz crystal with gold coating and mounted on a flow cell.

In some embodiments, the bacteria are detected by detecting the bacterial nucleic acid. In some embodiments, the complexes are separated from the incubation mixture of the bacteriophage and the bacteria. The nucleic acid of the complex is isolated. The isolated nucleic acid will comprise bacterial nucleic acid and nucleic acid from the bacteriophages that specifically infect these bacteria. Bacterial DNA is large and supercoiled and plasmid DNA is supercoiled. In contrast, DNA from bacteriophage family Caudovirales is linear dsDNA. Thus, bacteriophage DNA can be separated from bacterial DNA due to the difference in size and morphology.

In some embodiments, the detection of bacterial nucleic acids is by mass spectrometry. Nucleic acid is extracted from a sample comprising bacteria and/or complex. The extracted nucleic acid is concentrated and applied on a solid support. The nucleic acid is dried. The mass spectra from the purified nucleic acid sample are recorded on a mass spectrometer device. The mass spectra of a standard healthy cells and phage infected cells are compared. A difference is indicative of phage infected bacteria.

In some embodiments, the bacteria are detected by using a calorimetric device. The endothermic and exothermic peaks of the negative control (uninfected bacteria) and the sample suspected of comprising a complex of bacteriophage and bacteria are obtained from thermograms of the calorimetry output. The peaks are compared to infer phage infected bacteria. In some embodiments, the onset temperatures of the endothermic or exothermic peaks are studied. In some embodiments, the total enthalpy of the sample is used to infer the presence of phage infected bacteria.

In some embodiments, the bacteria are detected by fluorescence. In some embodiments, the bacteria and/or the bacteriophage comprise a detectable label. In some embodiments, the bacteria and/or the bacteriophage comprise more than one detectable label, e.g., at least two, at least three detectable labels. In some embodiments, the detectable labels are different. In some embodiments, the detectable label is a fluorescent moiety. In some embodiments, a fluorescent moiety can be stimulated by a laser with the emitted light captured by a detector. The detector can be a charge-coupled device (CCD) or a confocal microscope, which records its intensity.

In some embodiments, the bacteria are detected by the change in the physiological and chemical changes due to the complexation by enzymatic reaction. In some embodiments, the bacteria are detected by the color change due to enzymatic reaction, e.g., beta galactopyranoside assay, glucuronidase, N-acetyl-b-galactosidase, esterase, glucosidase, and lactate dehydrogenase (LDH).

### EXAMPLES

### Example 1

### Immobilization of a Complex of Bacteriophage and Bacteria on Beads

Preparation of S1 Host Bacteria

Bacterial host cell, S1 was washed twice with 1× PBS pH 7.4 buffer to remove MRS media. SYBR® green (Invitrogen) stain was added to a final concentration of 2×.

Preparation of PMA Stained J1 Phage

One mL of J1 phages (10⁸ PFU/ml) were concentrated using 50 kDa Amicon ultrafiltration filter (EMDmillipore, Billerica, Mass.). Spins were conducted at 7000 rpm for 5 minutes on a tabletop centrifuge. J1 phage was recovered in 400 μL. To the concentrated J1 phage, 2.5 μL of 20 mM PMA stain was added.

Preparation of Beads

1) 200 μL of BioMag (51 mg/ml) amine beads (Bangs Laboratory, Fishers, Ind.) were washed with 1× PBS pH 8.0 buffer. Beads were resuspended in final volume of 200 μL 1× PBS pH 8.0 and served as the stock beads for the following experiment.

2) To prepare BS3 (bis(sulfosuccinimidyl)suberate), (Thermo Fisher Scientific) linked magnetic beads, 5 μl of stock beads was dispensed into 200 μL of 1× PBS pH 8.0. Then, 20 μL of 70 mM solution of BS3 was added to the beads and allowed to incubate for 45 minutes at room temperature on a rotating wheel. The BS3 linked beads were equally split into two 100 μL portions and then placed on a magnetic rack for 3 minutes. The solution containing excess BS3 linker was removed from the beads. In order to attach J1 phage to the linked beads, 200 μL of 10⁹ PFU/ml J1 stained with PMA was added to one sample of beads and incubated at room temperature for 50 minutes on a rotating wheel.

3) The control set were non BS3-linked magnetic beads and these were prepared by dispensing 5 uL of stock magnetic beads into 200 uL 1× PBS pH 8.0. The sample was split into two 100 uL portions and placed on the magnetic rack to remove the buffer. J1 (200 uL of 10\hat{ }9 PFU/ml) stained with PMA was added to one of the beads.

4) Excess and unreacted linkers were quenched by adding 10 μL of 1M Tris HCl, pH 7.5 to all four samples and incubated for 15 minutes at room temperature. The samples were placed on a magnetic rack to remove the solution.

5) 500 μL MRS was added to all 4 samples to block remaining open sites on the bead for 1 hour.

6) Beads were washed twice with 1× PBS and ready to use.

Addition of S1 to Prepared Magnetic Beads

The SYBR® green stained S1 bacteria (200 μL of 10⁸ cells) was added to each of the four bead samples and incubated with for 5 minutes at room temperature. The beads were then washed four times with 500 μL 1× PBS, pH 7.4 to remove unbound bacteria and resuspended in a final volume of 90 μL 1× PBS.

Results:

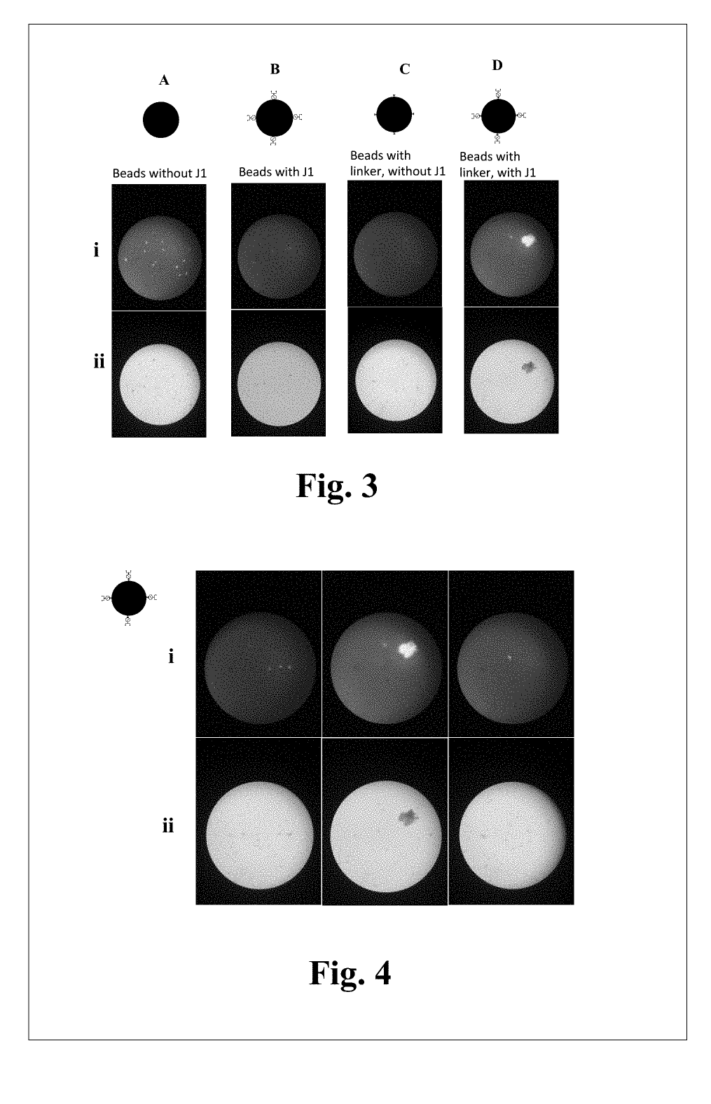

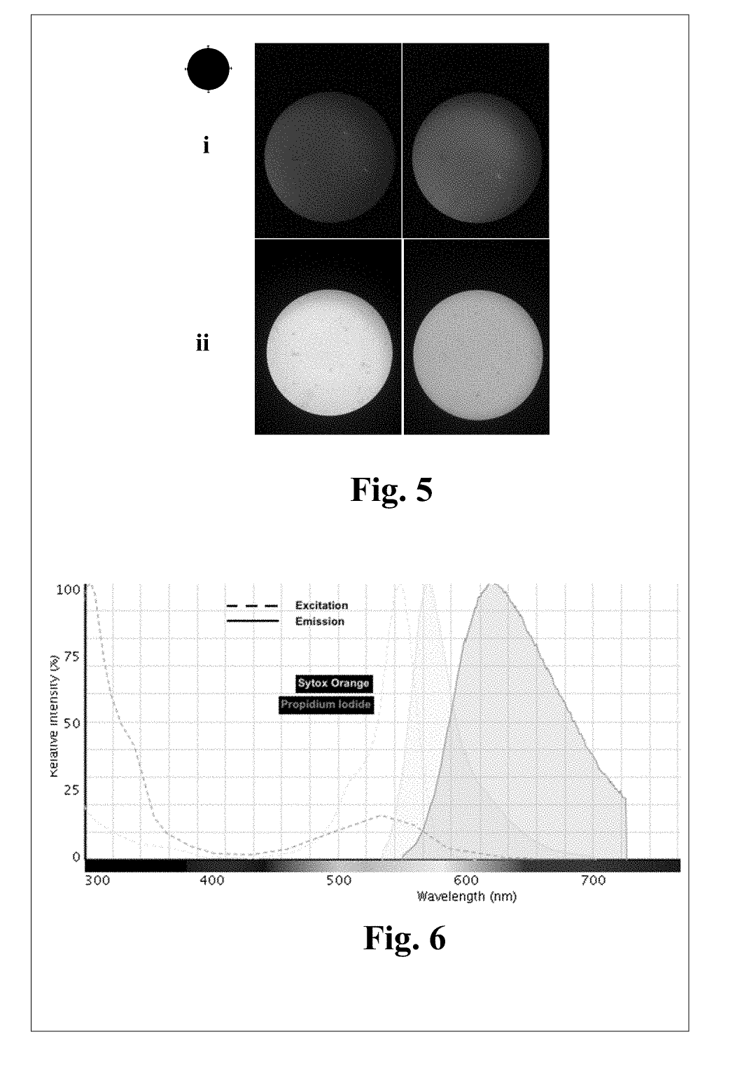

Non-BS3 linked magnetic beads with passive adsorption of J1 (PMA) onto the magnetic beads: Did not show signs of bacteria binding to the beads (FIG. 3, Col. B, Row (i) (fluorescence image) and Row (ii) (bright field image)).

Non-BS3 linked magnetic beads without exposure to bacteriophage J1 (PMA stained) had low levels of Si bacteria binding to the beads. Most of the beads were without bacteria (FIG. 3, Col. A, Row (i) (fluorescence image) and Row (ii) (bright field image)).

BS3-linked magnetic beads comprising the linkers but without immobilizing bacteriophage J1 (PMA stained) had very low levels of Si bacteria bound to the beads. Most of the beads were without bacteria (FIG. 3, Col. C, Row (i) (fluorescence image) and Row (ii) (bright field image)).

Bacteriophage J1 (stained with PMA) immobilized to magnetic beads with the BS3-linker showed the most bacteria bound compared to the above three samples. Even though most individual beads were not coated in bacteria, there were clumps of magnetic beads that had bound bacteria (FIG. 3, Col. D, Row (i) (fluorescence image) and Row (ii) (bright field image).

### Example 2

### Preferential Labeling of DNA with Nucleic Acid Binding Dyes

The preferential labeling of nucleic acid was tested with propidium monoazide (PMA), propidium iodide (PI) and SYTOX® Orange dyes. Permeable bacterial cells were initially stained with one of the three dyes and then stained with the other two dyes individually to test the blocking of the nucleic acid or displacement of the dyes from the nucleic acid.

0.6 ml of permeable S1 bacterial cells (10⁸ cfu/ml) were centrifuged at 13000 rpm. The supernatant was removed and the pellet was resuspended in 1 mL PBS. This wash step was employed to remove the media and cell debris.

In one set, 1-15 μL PMA (10 mM) was added to the washed cells and incubated in the dark or under light for 5-15 min. In some cases, the sample was exposed to light for 15-30 minutes. The bacterial cell solution was equally divided into two parts. To each bacterial cell solution, SYTOX® Orange (10 mM) and PI (10 mM) were added, respectively. The fluorescence was measured at different wavelength of excitation and emission corresponding to SYTOX® Orange, PI and PMA dyes to observe the effect of the second dye on the presence of PMA bound to the nucleic acid.

In another set, SYTOX® Orange (10 mM) was added to the washed cells and incubated in the dark or under light for 5 min. The bacterial cell solution was equally divided into two parts. To each bacterial cell solution, of PMA (10 mM) and PI (10 mM) were added, respectively.

In another set, PI (10 mM) was added to the washed cells and incubated in the dark or under light for 5-15 min. The bacterial cell solution was equally divided into two parts. To each bacterial cell solution, PMA (10 mM) and SYTOX® Orange (10 mM) were added, respectively.

The fluorescence was measured at different wavelength of excitation and emission corresponding to SYTOX® Orange (530/577 nm), PI (492/600 nm, 20 nm emission bandwidth) and PMA (492/600 nm, 80 nm emission bandwidth) dyes to observe the effect of the second dye on the presence of PMA bound to the nucleic acid. Exemplary excitation and emission spectra of SYTOX orange and propidium iodide dyes are shown in FIG. 6. The results are shown in Table 4 below.

TABLE 4
Blocking and Displacement of the Dyes

| | Excitation/Emission | | | |
|---|---|---|---|---|
| | Wavelengths | | | Observation |
| S1 without stain | 16 | 33 | 46 | |
| S1 with SYTOX ® | 1514 | 288 | 816 | leak of |
| orange (SO) | | | | SYTOX ® |
| | | | | signal into PI |
| | | | | and PMA |
| Add PMA to the | 21 | 58 | 137 | PMA added |
| SYTOX ® stained | | | | after staining |
| S1 | | | | with |
| | | | | SYTOX ® |
| | | | | PMA |
| | | | | displaces |
| | | | | SYTOX ® |
| | | | | decreasing the |
| | | | | fluorescence |
| | | | | of SO and |
| | | | | increasing |
| | | | | PMA |
| | | | | fluorescence. |
| | | | | Low signal in |
| | | | | PMA channel |
| | | | | is because |
| | | | | efficiency of |
| | | | | PMA is low |
| Add PI to the | 143 | 136 | 300 | PI displaces |
| SYTOX ® stained | | | | SYTOX ® |
| dye | | | | |
| S1 with PI | 50 | 109 | 240 | |
| Add SYTOX ® | 158 | 139 | 317 | some |
| Orange to PI | | | | displacement |
| stained S1 | | | | but not |
| | | | | significant |
| Add PMA to PI | 22 | 58 | 150 | PMA |
| stained S1 | | | | displaces PI. |
| | | | | Fluorescence |
| | | | | of PI is |
| | | | | lowered with |
| | | | | PMA addition |
| S1 with PMA | 20 | 57 | 129 | |
| Add PI to PMA | 32 | 62 | 135 | No significant |
| Stained S1 | | | | change to |
| Add SYTOX ® | 31 | 48 | 126 | PMA |
| Orange to PMA | | | | fluorescence. |
| Stained S1 | | | | PMA is not |
| | | | | displaced by |
| | | | | PI or |
| | | | | SYTOX ® |
| | | | | Orange. The |
| | | | | PMA is a |
| | | | | strong |
| | | | | binder/blocker |

### Example 3

### Testing of Beads for Use with SYROX Orange® Dye

Magnetic beads and polystyrene beads of 1.5 μm in diameter were incubated with SYROX Orange® Dye. Excess dye was washed off and fluorescent readings were measured in photons per second.
1. 5μM SYTOX® Orange to .5% beads—Magnetic with NH2+ functionality and polystyrene with NH2+ functionality. The magnetic beads were washed by rinsing 1× PBS. A magnetic rack was used to separate the magnetic beads from solution, and remove all the liquid from the tube. Washing with 1× buffer was repeated three times.
To wash polystyrene beads:
a. Beads were loaded in to a 100K amicon centrifuge filter,
b. Centrifuged for 5 min at 10,000 rpm.
c. Beads were rinsed with 1× buffer. Centrifuged for 5 min at 10,000 rpm. Washing with 1× buffer was repeated three times. 3. The beads were resuspended in original volume. Signal was read with detector.

The results were shown in Table 5 below.

TABLE
Non-specific binding of SYTOX ® orange dye to beads

| | | MB 1.5 um w | | PS 1.5 um w |
|---|---|---|---|---|
| Trial | MB 1.5 um | SYTOX ® | PS 1.5 um | SYTOX |
| 1 | 703.25 | 771 | 1303.5 | 59917 |
| 2 | 737.41 | 887.76 | 1499.29 | 67001.18 |
| 3 | 724.24 | 919.06 | 2229.65 | 56816 |
| Average | 722 | 859 | 1,677 | 61,245 |
| Stdev | 17 | 78 | 488 | 5,221 |

SYTOX ® orange dye binds more non-specifically to polystyrene beads.

### Example 4

### Detection of Bacteriophages by Preferential Labeling of DNA with Nucleic Acid Binding Dyes

A bacterial based assay was developed to capture host-specific bacteriophages. The captured bacteriophages are stained with SYTOX® Orange (Molecular Probes) and SYTOX® Orange has fluorescence enhancement of >500-fold upon binding DNA. SYTOX® orange is a cell impermeant nucleic acid dye that stains compromised cells. In every batch of healthy cells, there exist a percentage of dead cells due to the natural life and death cell cycle. This causes unpredictable background fluorescence due to dead cells.

In order to address the dye entering normal dead cells found in all bacterial samples, a protocol was developed using commercially available nucleic acid stains to preferentially block the signal from compromised bacteria and allow us to only detect bacteriophage.

Propidium monoazide (PMA, Biotium, Fremont, California) (AB/Em, 510/610) is a cell impermeant, fluorescent dye that forms covalent bonds to nucleic acids of compromised cells. By strategically staining the bacteria-phage complex, bacteriophages can be detected specifically.

Bacterial cells were washed with PBS to remove media and cell debris before staining with PMA. The PMA dye preferentially stains compromised bacterial cells and is classified as cell impermeant. The bacterial cells are used as the capturing vehicle to attract host specific bacteriophages. Non-specific bacteriophages do not bind to bacteria and are washed away after pelleting the bacteria and a wash step.

SYTOX® orange or another similar cell impermeant dye is used to stain the nucleic acids of the bacteriophages. PMA continues to block the SYTOX® orange from intercalating with DNA in compromised bacterial cells. Therefore the total signal output is from captured phages.

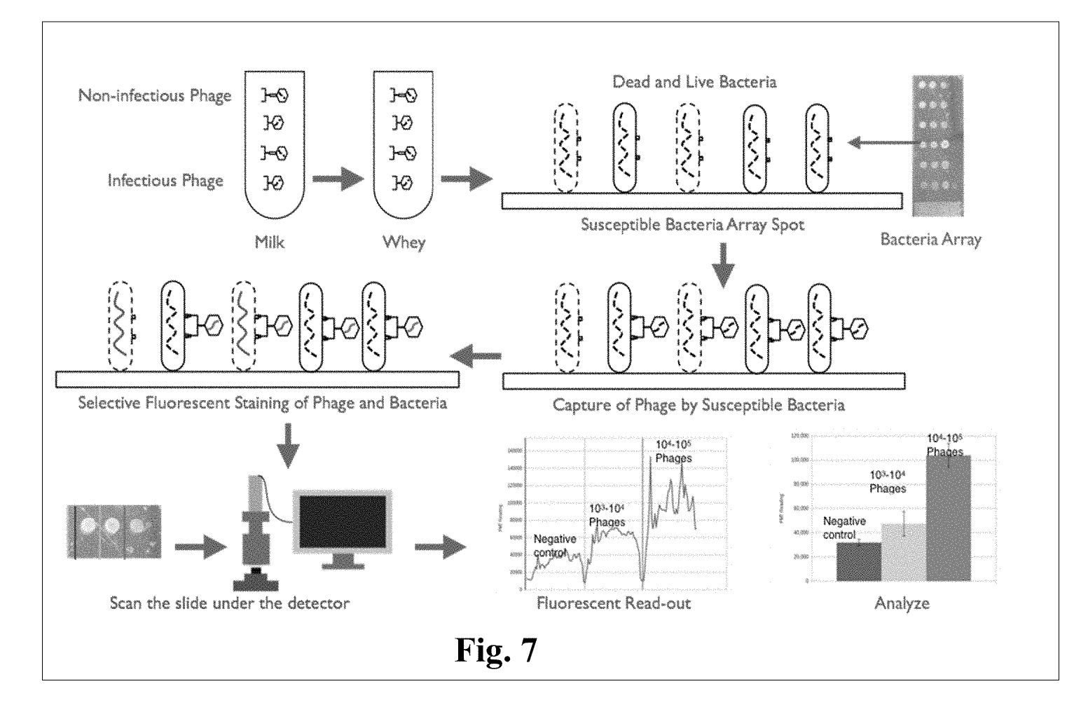

Briefly, 0.5 ml of bacterial cells, (OD 0.1-0.9) centrifuged in 1.5 ml tube for 13 k rpm for 1 min. The supernatant was removed and the cell pellet was resuspended in 1 mL buffer to wash the cells. The wash step was repeated to remove the media and cell debris. 5-10 uL PMA (1-10 mM) was added to the washed cells and incubated in the dark for 5 minutes. The sample was exposed to light for 15 minutes to allow the covalent linking of PMA to the nucleic acid. The PMA labeled bacteria were ready to be used as the capturing vehicle of host-specific phages. Following the capture of the specific bacteriophages, 1-10 uM of SYTOX orange or another cell impermeant dye was used to stain the captured phages. The fluorescent signal was detected to detect the captured bacteriophages. The results are shown in FIG. 7. Bacteriophages at a concentration of 10³-10⁴ pfu/μ1. Higher signals are observed when bacteriophages at a concentration of 10⁴-10⁵ pfu/μl were used.

### Example 5

### Raman Spectroscopy and Surface Enhanced Raman Spectroscopy(SERS) for Bacterial Detection

For SERS internal mode, harvested bacterial cells are resuspended in AgNO3 solution (1 M) and incubated for 5 min. The cells are then collected and are washed twice in water by centrifugation at 4,500 g for 10 min at 4° C. Afterwards, the cells are resuspended in NaBH4 solution (0.5 M) for further analysis. For SERS external mode, harvested bacterial cells are first resuspended in NaBH4 solution and incubated for 5 min. The cells are then collected by centrifugation and resuspended in AgNO3 solution for further analysis. In a typical experiment, the bacterial cells are treated with 0.1% Triton X-100 (v/v) for 5 min, prior to the assay. Then, 10 μL of such treated bacterial suspension (˜1×10⁶ CFU/mL) are mixed with 10 μL of NaBH4 (0.5 M). The mixture is then incubated for 3-5 min. Subsequently, 800 μL of AgNO3 (1 M) are pipetted into the mixture followed by vortexing.

Raman analysis is conducted by addition of 5 μL of the bacterial suspensions obtained from above modes onto the surface of glass cover slips (0.13 mm thickness, 15 mm diameter, Ted Pella Inc., Redding, Calif., USA). SERS spectra are recorded on a benchtop Raman spectrometer (Sierra Snowy Range 785 series, Laramie, Wy., USA) under excitation wavelength of 785 nm. The exposure time is is and the number of accumulations for each measurement is 10. The spectral data are acquired over a Stokes Raman shift of 400-2,000 cm-1. For each study, three biological replicates are analyzed. To validate the results of the strain level discrimination, additional replicate is also analyzed.

### Example 6

### Electrochemical Detection of Bacteria

Protocol

Functionalization of Electrochemical Sensors

Thiolated capture probe is prepared at a concentration of 0.05 μM in 300 μM 1,6-hexanedithiol (HDT), 10 mM Tris-HCl, pH 8.0, 0.3 M NaCl, 1 mM EDTA and is incubated in the dark at room temperature for 10 min. Incubation of the thiolated capture probe with HDT ensures that the thiol group on the capture probe is reduced, resulting in more consistent results.

A stream of nitrogen is applied to bare gold 16 sensor array chip(s) for 5 sec to remove moisture and/or particulates. A 6 μl of the HDT-thiolated capture probe mix is applied to the working electrode of all 16 sensors of the sensor array and store the sensor chip(s) in a covered Petri dish at 4° C. overnight. Thiolated capture probes bind directly to the bare gold electrode and the HDT acts to prevent overpacking of the capture probes and keep them in an extended conformation that promotes hybridization with the target.

The following day, the sensor chip is with deionized H2O for 2-3 sec and dry under a stream of nitrogen for 5 sec. A 6μl of 10 mM Tris-HCl, pH 8.0, 0.3 M NaCl, 1 mM EDTA, 1 mM 6-mercapto-1-hexanol (MCH) is applied to the working electrode of all 16 sensors and incubate for 50 min. This and all subsequent sensor chip incubations are performed in a covered Petri dish at room temperature. MCH acts as a blocking agent, filling in any gaps where the thiolated capture probe or HDT is not present on the electrode surface.

Sample Preparation

One ml of bacterial culture is transferred in the log phase of growth (OD600≈0.1) to a microcentrifuge tube and centrifuge at 16,000× g for 5 min. The culture supernatant is removed. The bacterial pellet can be processed immediately or can be stored at −80° C. for later use. The bacterial pellet is thoroughly resuspended in 10 μl of 1 M NaOH by applying the pipette tip to the bottom of the microcentrifuge tube and pipetting up and down several times. Incubate the suspension at room temperature for 5 min. The bacterial lysate is neutralized by adding 50 μl of 1 M Phosphate Buffer, pH 7.2, containing 2.5% bovine serum albumin (BSA) and 0.25 mM of a fluorescein-modified detector probe. The neutralized lysate is incubated for 10 min at room temperature. Fluorescein-modified detector probes hybridize with bacterial rRNA target molecules.

Electrochemical Sensor Assay

The MCH is washed from the sensor chip with deionized H2O for 2-3 sec and is dried under a stream of nitrogen for 5 sec. A 4 μl of neutralized bacterial lysate is applied to the working electrode of each of 14 sensors and incubate for 15 min. Target-detector probe complexes hybridize to immobilized thiolated capture probes. A 4 μl of 1 nM bridging oligonucleotide in 1 M Phosphate Buffer, pH 7.2, containing 2.5% BSA and 0.25 μM fluorescein-modified detector probe is applied to 2 positive control sensors (used for signal normalization) and is incubated for 15 min. The sensor chip is washed with deionized H2O for 2-3 sec and is dried under a stream of nitrogen for 5 sec. A 4 μl of 0.5 U/ml anti-Fluorescein-HRP in 1 M Phosphate Buffer, pH 7.2, containing 0.5% casein is applied to the working electrode of all 16 sensors and is incubated for 15 min. The anti-Fluorescein-HRP binds to the immobilized fluorescein-modified detector probes. The sensor chip is washed with deionized H2O for 2-3 sec and is dried under a stream of nitrogen for 5 sec. A film well sticker is applied to the surface of the sensor chip and load into the sensor chip mount. A 50 μl of TMB substrate is pipette onto all 16 sensors and close the sensor chip mount.

Amperometry and cyclic voltammetry measurements are obtained for all 16 sensors using the Helios Chip Reader. Amperometric current is proportional to the rate of TMB reduction on the sensor surface.

### Example 7

### Detection of Bacterial DNA by Mass Spectrometry

Mass spectra are acquired using an Ultraflex I MALDI-TOF mass spectrometer (Bruker Daltonics, Bremen, Germany). Alternatively, a simpler MALDI-TOF instrument such as the benchtop Microflex (Bruker Daltonics) can be used without losing data quality. Measurements in linear positive ion detection mode are performed, using a Nd:YAG laser at maximum frequency of 66 Hz. Pulsed ion extraction (PIE) is set to zero. Acceleration voltage (IS1) is set to 20 kV. The mass range of spectra is from 2,000 to 20,000 m/z. The final resolution in the mass range of 7,000-10,000 m/z is optimized to be higher than 600 and absolute signal intensities are about 103. Automated spectrum acquisition is performed using the Auto Execute software with fuzzy control of laser intensity. At least 10⁷ bacterial cells are required for high quality mass spectra. For reference spectra six spots on the MALDI target are measured. On each spot, four spectra with 10 times 100 laser shots are accumulated. Twenty spectra are stored for the reference spectra library. For identification spectra is acquired by accumulating 1000 laser shots in ten 100 shot portions.

Factors influencing the intensities of signal peaks comprise concentration and location of proteins in the bacterial cell and biophysical properties of proteins such as solubility, hydrophobicity, basicity, and compatibility with MALDI. In general, most of the proteins detected by MALDI protein bacterial profiling derive from highly abundant, basic ribosomal proteins.

Data analysis: Mass spectra are analyzed with Flex Analysis software 2.4 (Bruker Daltonics). The mass spectral input data can be listed in generic data formats such as the extensible markup language (XML) to make them independent from the hardware used. Spectra are pre-processed using default parameters for reference spectra libraries also known as call main spectra libraries (MSPs). A maximum of 100 peaks with a signal-to-noise (S/N) ratio of 3 are selected in the range of 3,000-15,000 Da. Afterwards the main spectra are generated as a reference using all spectra given for a single microorganism. In general, 75 peaks are picked automatically, which occur in at least 25% of the spectra and with a mass deviation of 200 ppm.

For the evaluation of mass spectra reproducibility, the spectra are loaded into the ClinProTools 2.1 software (Bruker Daltonics). Through this process mass spectra are firstly normalized before baseline subtraction, peak detection, realignment, and peak-area calculation are applied. The optimal settings resulted in an S/N ratio of 5, a Top Hat baseline subtraction with 10% as the minimal baseline width, and a 3-cycle Savitsky-Golay smoothing with a 10 Da-peak width filter. For the example shown in FIG. 3 the coefficient of variation (CV) of each of the individual peak areas is determined; 100 peaks are taken for intra run assessment detected in 18 measurements and 75 peaks for inter run detected in 5 biological replicates. The mean CV for all of the signals from the same replicate sample is calculated to provide a measure of intra- and inter-run reproducibility.

### Example 8

### Detection of Bacteria by Atomic Force Microscopy

A Nanoscope IIIA AFM (Digital Instruments, Santa Barbara, Calif., USA) operating in contact mode in air is used to image cells and to measure interaction forces. The relative humidity was 50-60% and no a spring constant of k50.06 N/m (Digital Instruments). The radius of curvature of the AFM tip is approximately 50 nm. The Digital Nanoscope software (version 4.23) is used to analyze the topographic images of the surface, as well as the force—distance over the sample surface. During the force—distance measurements, the scanning rate in z-direction is maintained at 30 Hz. Each map of sample surface consisted of 64 3 64 grid points.

### Example 9

### Detection of Bacteria by Near-Field Scanning Optical Microscope

NSOM measurements were acquired using a SNOM 210 in the beta-type of the commercially available standard instrumentation equipment with a piezo scanning unit integrated into a microscope condenser (Carl Zeiss Jena GmbH, Digital Instruments Veeco GmbH). Micro-fabricated probes with silicon nitride tips coated with aluminum, with a typical aperture of 100nm, are mounted in a shear-force sensor support. The instrument is equipped with an argon ion laser (1=458 nm, 488 nm) and two HeNe lasers (1=543 nm, 633 nm) for near field illumination. The illumination intensity is independently tuned by an AOTF (Acousto Optical Tunable Filter). The laser light is coupled into the NSOM tip by glass fibers. The topographic scan is controlled by modulation of the lateral shear-force oscillation of the NSOM probe. Fluorescence and absorption are detected in air by an Achroplan long distance objective 40×/NA 0.6corr. and transferred to a photo-multiplier or an avalanche photodiode, respectively, using appropriate filter settings. The instrument is controlled by the NanoScope IIIc controller. The scans are performed using a probe velocity of less than 1 μm/s. Images 1×1 μm2 up to 10×10 μm2 are registered and visualized in 3D topographic false color plots using the NanoScope Ma software (version 4.42r1) running under Windows on a PC.

### Example 9

### Detection of Bacteria by Calorimetry

Samples (10-15 mg) are weighed to 0.01 mg, sealed in volatile aluminum pans and heated in a Perkin-Elmer DSC-2C at a rate of 10° C. min⁻¹ from about 10° C. to 120° C. Samples are weighed before and after calorimetric measurements to check for loss of mass during heating and the results of samples showing signs of leakage are discarded. An empty pan is used as a reference and, after heating, the sample is rapidly cooled to its initial temperature. Selected samples are then re-run in the DSC to investigate the reversibility of the thermograms. The sample dry mass is determined by piercing the pan and drying it overnight in an oven at 105° C. Data are collected and the calorimeter is controlled with a Perkin-Elmer data station but thermogram scans are transferred to a VAX-11/750 computer for high-resolution plotting and peak area determined by trapezoidal integration. The differences between the data collected during the first run and those collected on re-running the DSC are proportional to that component of the specific heat capacity caused by irreversible processes taking place during the first heating. The difference thermogram is useful for precise determination of the onset temperature of irreversible denaturation. Temperature and power scales are calibrated according to the manufacturer's instructions using the melting of indium and ice as standards.

### Example 10

### Non-Specific Binding of the Fluorescent Dyes to Magnetic Beads and Blocking of Non-Specific Signals from Other Dyes by PMA

1% magnetic beads were washed and suspended in 1× PBS. luM SYTOX® Orange, 1× SYBR Green, or 0.4 mM PMA was added to the beads and the fluorescence at 577/488/600 nm were recorded. To the SYTOX® stained beads, 0.2 mM PMA was added, and the fluorescence recorded at 600 nm. The order of the addition of the SYTOX® Orange and PMA were reversed and the fluorescence recorded at the respective emission wavelengths.

| | | AMS40 (4-5 | |
|---|---|---|---|
| | Ex/Em | uM) | AM80 (8-10 |
| Treatment | Reading | Well, B1 | uM) Well, B3 |
| No dye | 492/600 | 27 | 47 |
| No dye | 530/577 | 28 | 28 |
| Add Sytox Orange to 1 | 530/577 | 103 | 52 |
| uM | | | |
| | 492/600 | 49 | 46 |
| Add PMA to 0.2 mM | 530/577 | 27 | 30 |

SYTOX ® Orange Dye (530/577);
PMA (492/600).

### Experiment II. Add PMA to Beads First, Then Add SYTOX® Orange.

| | Ex/Em | AMS40 (4-5 | AM80 (8-10 |
|---|---|---|---|
| Treatment | Reading | uM) Well, B2 | uM) Well, B4 |
| No dye | 492/600 | 30 | 34 |
| No dye | 530/577 | 21 | 26 |
| Add PMA to 0.2 mM | 530/577 | 24 | 27 |
| | 492/600 | 39 | 50 |
| Add Sytox Orange to 1 | 492/600 | 45 | 49 |
| uM | | | |
| | 530/577 | 26 | 30 |

Sytox Orange Dye (530/577);
PMA (492/600).

Experiment III. Test Absorbance, Scattering of Dyes with Beads

| | Sybr Green | PMA | Sytox Orange |
|---|---|---|---|
| Beads | 492/530 | 492/600 | 530/577 |
| Bead 1 | 19, 14, 14 | 42, 39, 37 | 27, 28, 23 |
| No Dye | | | |
| Bead 2 | 16, 25, 27 | 108, 106, 102 | 83, 92, 88 |
| With Dye | | | |
| Bead 2 | 11, 12, 8 | 44, 40, 38 | 29, 26, 33 |
| No Dye | | | |
| Bead 2 | 13, 12, 12 | 98, 96, 101 | 67, 68, 67 |
| With Dye | | | |
| Bead 3 | 15, 12, 10 | 29, 29, 32 | 22, 20, 21 |
| No Dye | | | |
| Bead 3 | 23, 8, 10 | 75, 77, 77 | 18, 17, 20 |
| With Dye | | | |

Thus, Results: PMA effectively blocks SYTOX® Orange from fluorescing.

### Example 11

### Detection of Bacteria by Monitoring the Reduction of Resazurin and Resorufin

To 1-1000 μl of samples (milk, whey, salt whey, culture, milk culture, broth), 1-1000 μl of bacterial cells with OD˜0.01-1 were added. In some cases, bacteriophages specific for the bacteria are added. In some embodiments, the bacteriophages were bound by a compound. To this mixture, Resazurin and/or Resorufin and/or Dihydroresorufin were added as a fluorescent tag. The fluorescence of Resorufin was measured between 27-37° C. In some experiments, the sample was incubated with the cells before the addition of the fluorescent tag. In some samples, the fluorescent tags were incubated with the sample. In some experiments fluorescent tags were incubated with cells. In some experiments, the incubated sample and cells were diluted one-fold before the addition of the tag. In some experiments, the incubated sample and cells were diluted one order before the addition of the tag. The fluorescence of Resorufin was measured at or above 577 nm over a period of time. The kinetic profile of the reduction of Resazurin to Resorufin to Dihydroresorufin of bacterial cells infected with bacteriophage were compared to corresponding uninfected standard bacterial samples. The above conditions were tested in the following experiments.

1. Incubating Cultures and Bacteriophages Together at 32° C., and adding Resazurin to the Diluted and Undiluted Samples

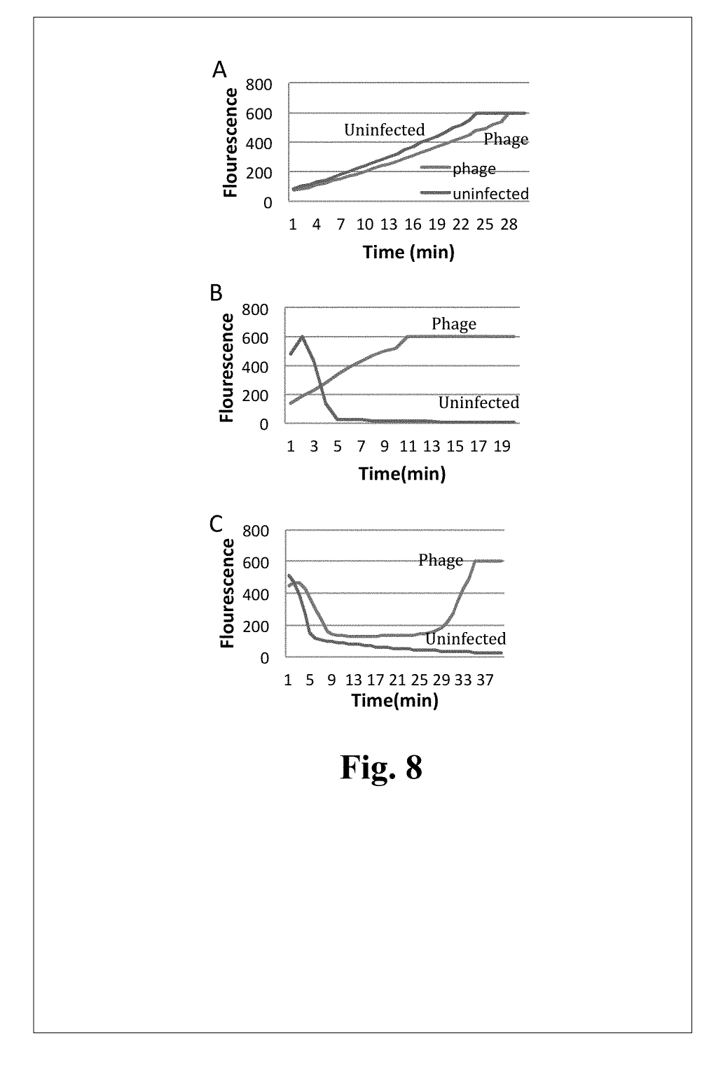

In one case, wild-type phages (10⁵ pfu/ml) and cocci and/or bacillus (gram positive) bacterial culture were added to milk. The mixture was incubated for 45 min. The incubation mixture was diluted 1-fold. To the diluted mixture, Resazurin was added and mixed. The absorbance and/or absorbance, scattering and/or fluorescent intensity of the mixture was measured at 577 nm for at least 30 min. The fluorescence intensity was plotted as a function of time as shown in FIG. 8A.

In another case, wild-type phages (10⁴ pfu/ml) and cocci and/or bacillus bacterial culture were added to of bacterial culture medium. The mixture was incubated for 90 min. To the incubation mixture, Resazurin was added and mixed. The absorbance and/or absorbance, scattering and/or fluorescent intensity of the mixture was measured at 577 nm for at least 20 min. The fluorescence intensity was plotted as a function of time as shown in FIG. 8B. Longer incubation times (75-120 minutes) resulted in the uninfected cells converting more of the Resorufin to Dihydroresorufin thus the infected cells had higher levels of fluorescence and the lines cross over.

In another case, wild-type phages (10³ pfu/ml) and cocci and/or bacillus bacterial culture were added to bacterial culture medium. The mixture was incubated for 75 min. To the incubation mixture, Resazurin was added and mixed. The absorbance and/or absorbance, scattering and/or fluorescent intensity of the mixture was measured at 577 nm for at least 40 min. The fluorescence intensity was plotted as a function of time as shown in FIG. 8C. Longer incubation times also increased sensitivity allowing the detection of bacteria with bacteriophage concentration as low as 10³ pfu/mL.

Additionally, inventors identified an unexpected and surprising difference in the kinetic profile of the reduction of Resazurin to Resorufin to Dihydroresorufin between phage infected bacterial cells and uninfected bacterial cells. The uninfected bacterial cells reduced the Resazurin (weakly fluorescent) to Resorufin (fluorescent) and finally to Dihydroresorufin (non-fluorescent) at a faster rate as compared to phage infected bacterial cells. The reduction of Resazurin to Resorufin is irreversible while the reduction of Resorufin to Dihydroresorufin is reversible. After some time, the predominant metabolic product of Resazurin is Dihydroresorufin (non-fluorescent) for uninfected bacterial cells. As a result, the fluorescence peaks faster for uninfected bacterial cells, and the fluorescence diminishes with time.

In contrast, the reduction of Resazurin (weakly fluorescent) to Resorufin (fluorescent) is slower for phage infected bacterial cells. As a result, the fluorescence peak for Resorufin appears later in phage infected bacterial cells. Additionally, phage infected bacterial cells preferably oxidize Dihydroresorufin (non-fluorescent) to Resorufin. Accordingly, using a phage specific for bacteria, a kinetic profile of infected bacteria is indicative of the presence of the bacteria in a sample.

2. Adding Resazurin at Different times of Incubation

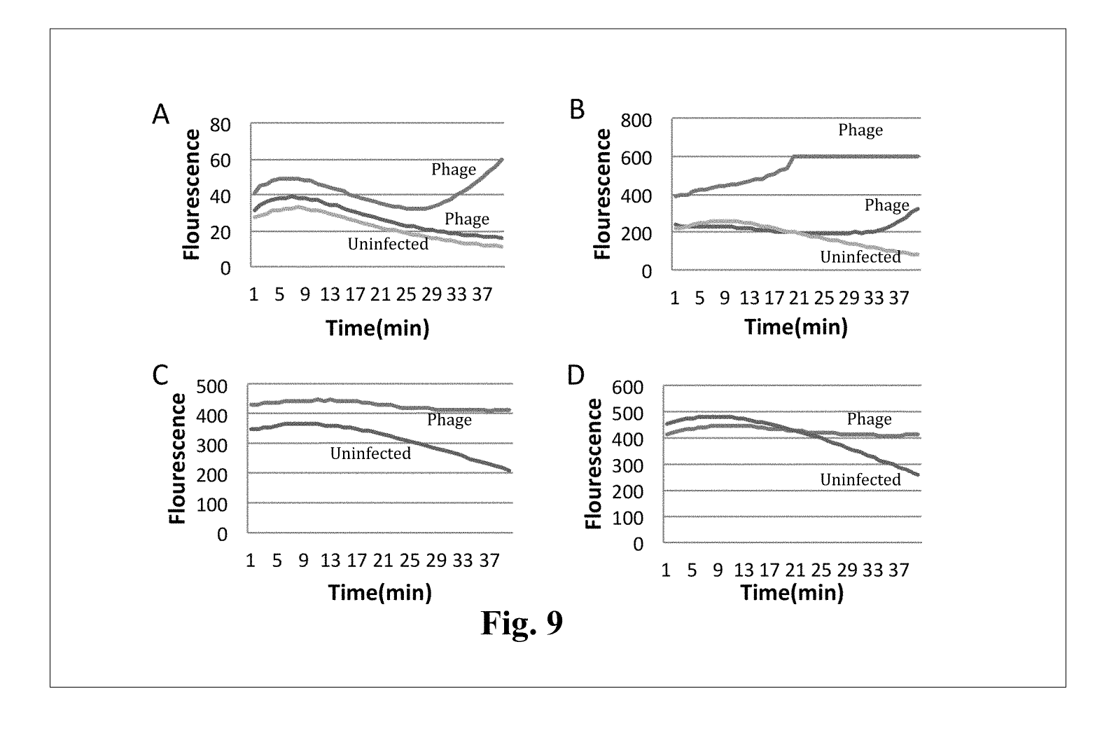

Adding Resazurin at the start of incubation (T0) resulted in an increased efficiency of the assay by reducing a step. The infected cells having higher levels of fluorescence compared to the uninfected control. All samples rapidly become pink as the Resazurin is reduced irreversibly to Resorufin. The sample then became white in color as the Resorufin is reduced to Dihydroresorufin, a reversible step. After this step occurs, based on the phage concentration, phage infected cell samples start to have an increased pink color (Resorufin) while readings on the uninfected controls continue to decrease. The results with different concentration phages are shown in FIG. 9A-D.

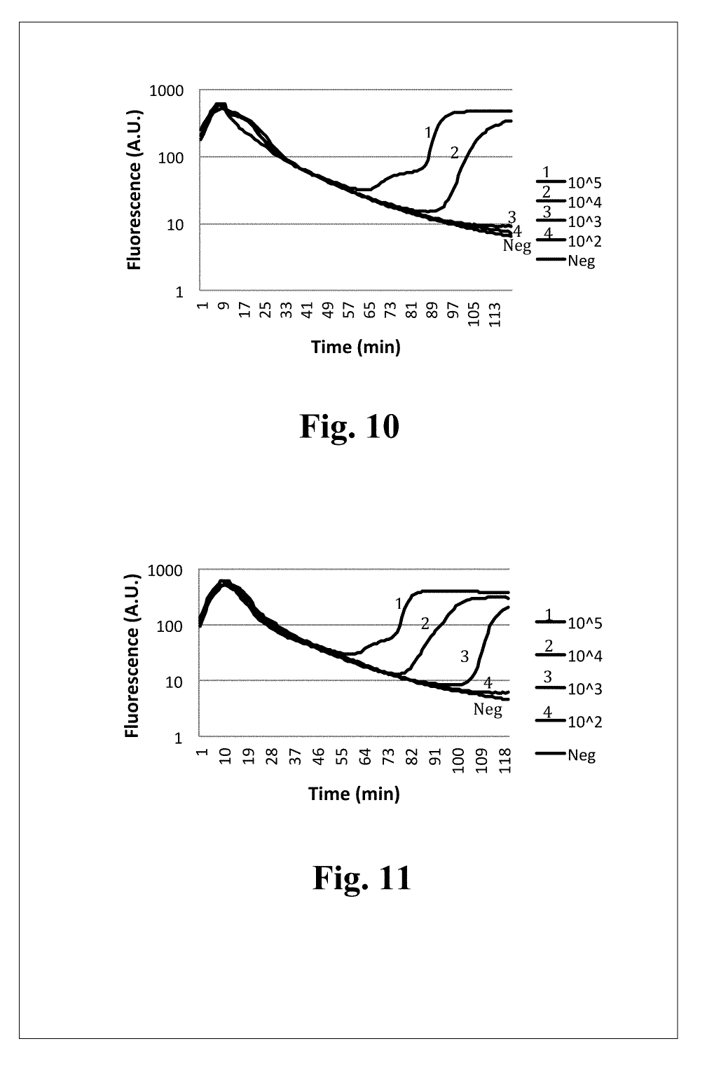

When varying concentration of wild-type phages (10⁵ pfu/ml-0 pfu/ml), bacterial culture (cocci and bacillus) and Resazurin was added and mixed with milk and incubated for 4 hours, the kinetic profile of the reduction of Resazurin was significantly different than of the phage infected cells as shown in FIG. 10.

3. Effect of Temperature on the Length of Time Required to Detect Infection of Bacteria with Phages.

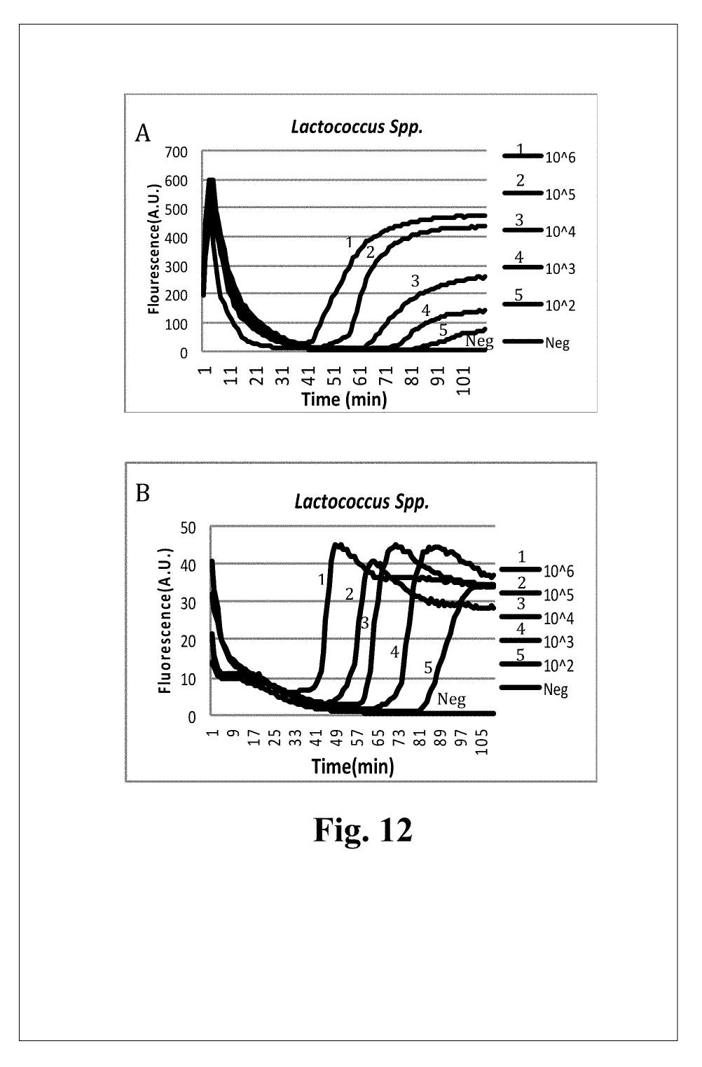

Increasing the temperature beyond 29° C. decreased the length of time needed to detect differences between the infected and uninfected cells by about 10-20 minutes and increased sensitivity, allowing detection of bacteria using bacteriophage concentration as low as 10² pfu/mL of phage (FIG. 11).

4. Difference Between Starting Incubations with Resorufin and Resazurin.

To see if the times to detect the bacteria could be decreased further, Resorufin instead of Resazurin was added to eliminate the time required for the cells to convert Resazurin to Resorufin. This did not significantly shorten the length of time to detection of phage infected bacteria using phage at higher concentrations but did shorten times by about 5-10 minutes for lower concentrations of virus. When the initial compound is Resazurin, the fluorescence and/or absorsance intensity of Resorufin shows a correlation with the phages. It is observed that in additon to the change in kinetics of change from Dihydroresorufin to Resorufin in the presence of infectious phages, the amount of Resorufin oxidised from Dihydroresorufin is proportional to the phage concentration when the reagent used is Resazurin. When the initial reagent is Resorufin, in some experiments, the final oxidized fluorescence shows little correlation to the amount of phage present (FIG. 12A-B).

Difference in Time to Detection of Bacteria with Different Bacteria and Phages.

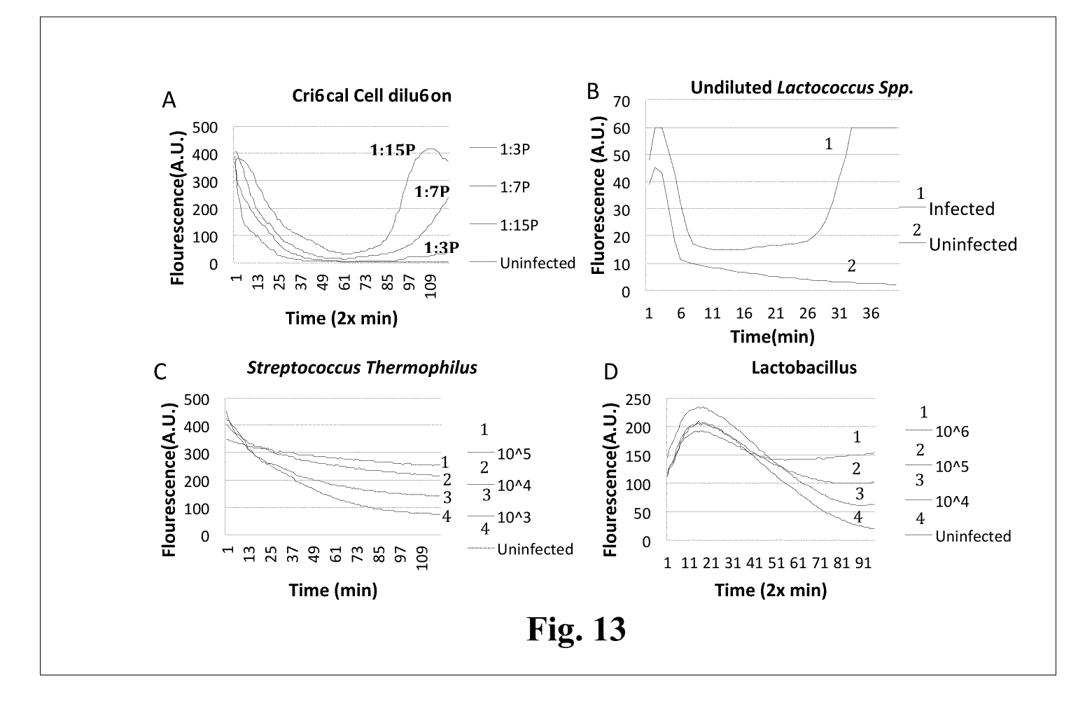

The time to detection of phage infected bacteria varied with different cells and phage. cocci and/or bacillus cells and wild-type phage required almost twice as long for the assay to detect phage infected cocci and/or bacillus bacteria as compared to different wild-type phage Detection of wild-type phage infection of the cocci and/or bacillus bacteria culture was also faster at 37° C. than 32° C. as shown in FIG. 13A-D.

Binding of PMA and PMAX to bacteriophage renders the phage non-lytic.

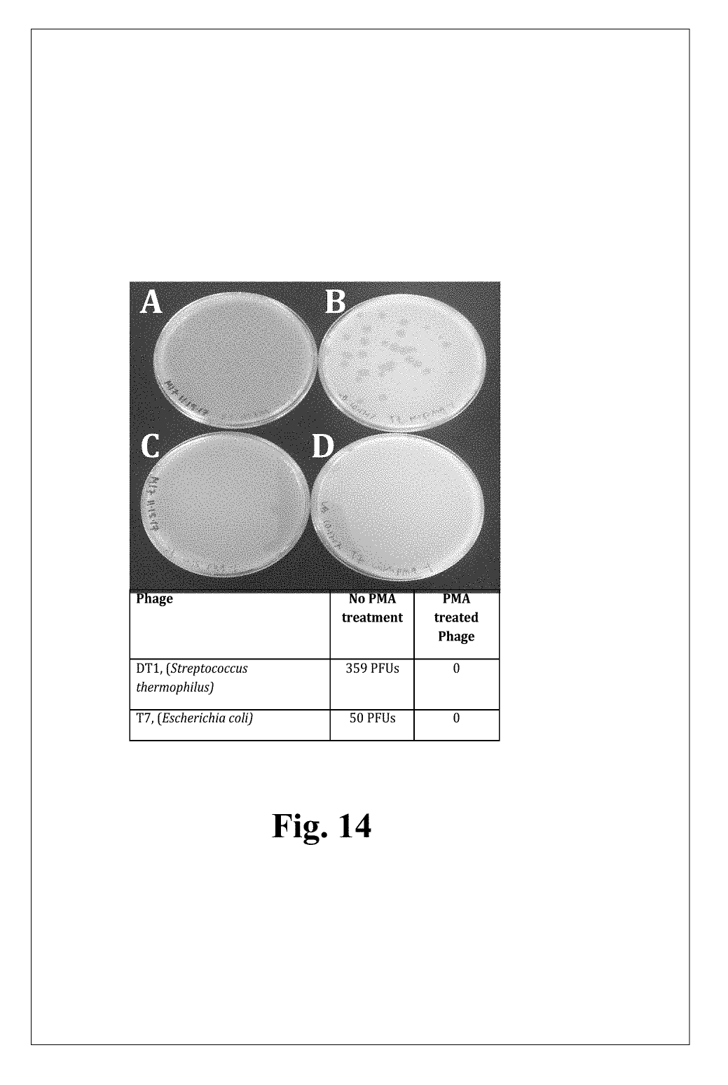

Two strains of bacteriophages, DT1 and T7 with and without PMA were tested for lytic activity. Streptococcus thermophilus with (FIG. 14A) untreated DT1 bacteriophage shows plaque formation and FIG. 14B shows PMA treated DT1 does not form plaques. Escherichia coli with (FIG. 14C) untreated T7 bacteriophage show plaque formation and PMA treated T7 does not form plaques (FIG. 14D). The plaque counts are tabulated in the FIG. 14

Resazurin Assay with E. coli Only E. coli with T7, and E. coli with PMA Treated T7.

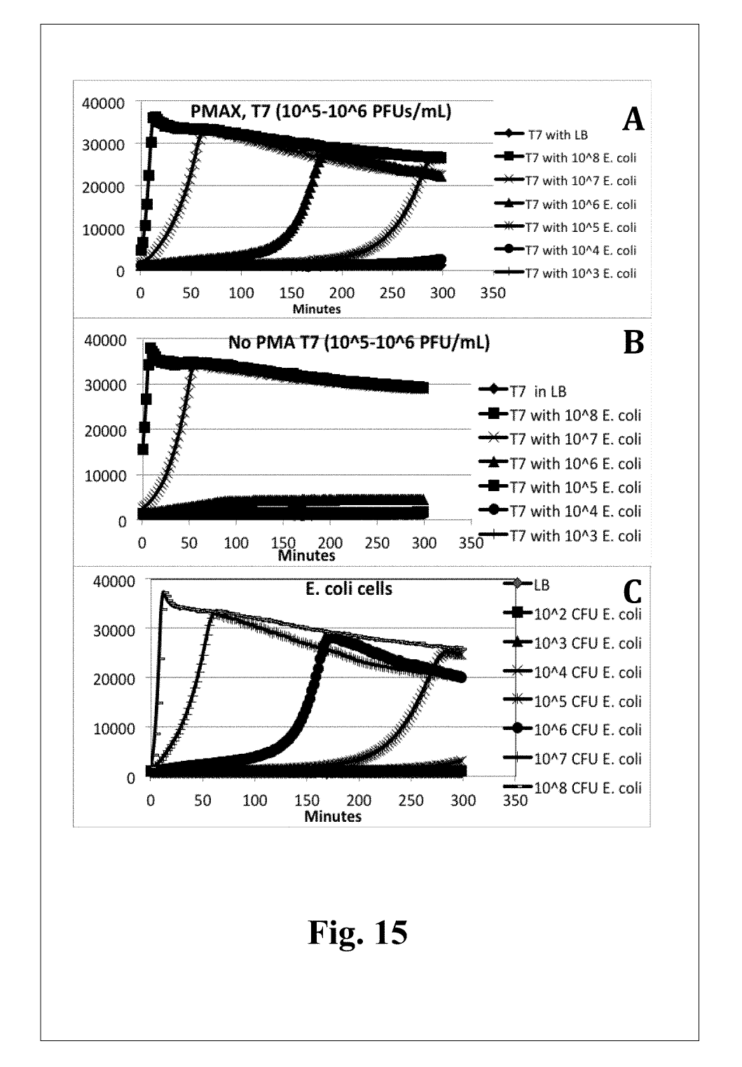

Biotinylated T7 bacteriophages were immobilized onto streptavidin magnetic beads. The T7-beads with (FIG. 15A) and without PMA (FIG. 15B) staining were incubated with E. coli. The captured E. coli was tested form viability with Resazurin. For comparison, viability of E. coli alone is shown in FIG. 15C.

Resazurin assay with capture of E. coli from a mixture of bacteria with T7 treated with PMA

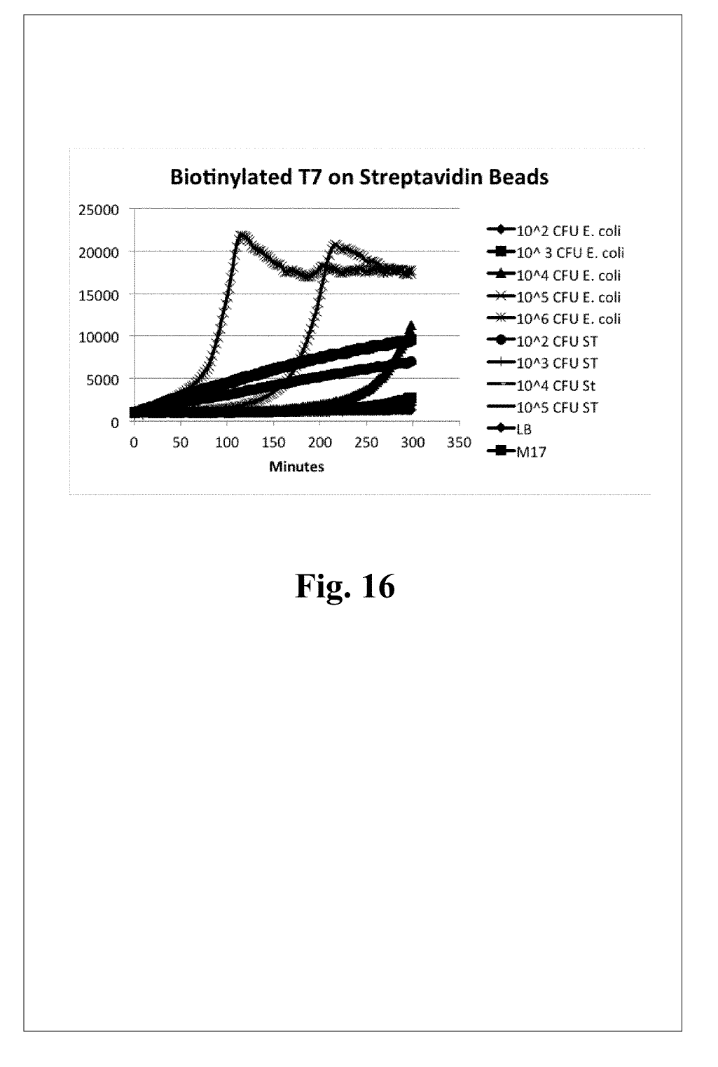

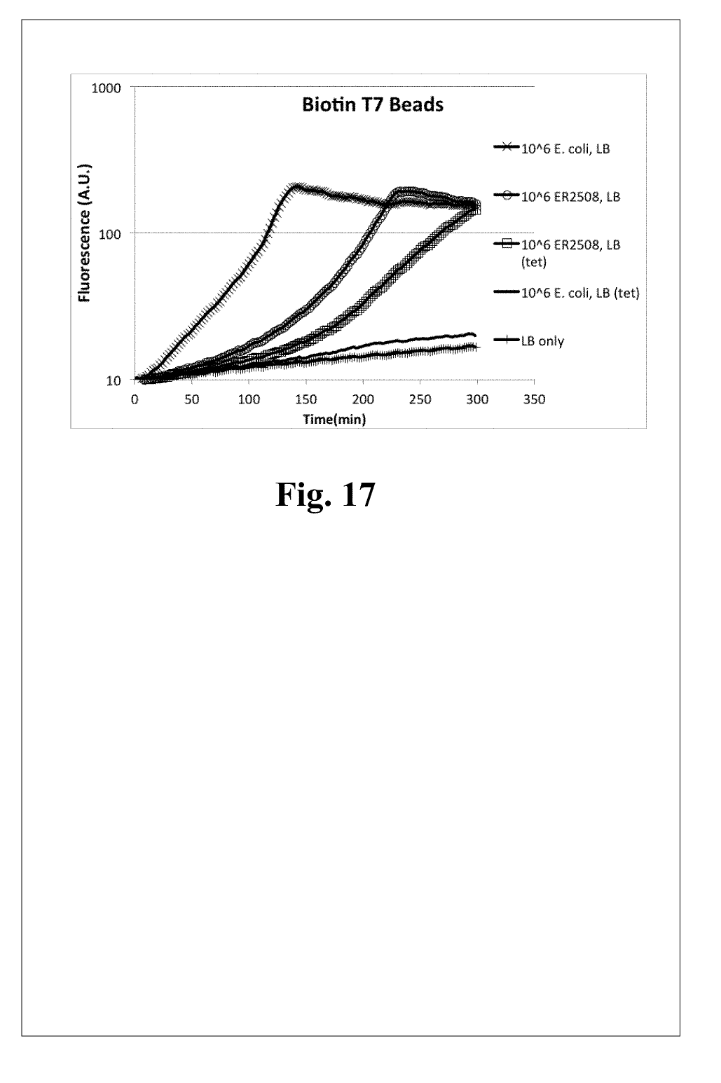

Biotinylated T7 bacteriophages were immobilized onto streptavidin magnetic beads. T7 beads were treated with PMA and was introduced into sample containing E. coli and Streptococcus thermophilus as a negative control. The beads were then washed to remove excess and unbound bacteria. After a 4.5-hour incubation for bacteria outgrowth, the binding and capturing ability of the T7 coated beads were measured using Resazurin. The results shown in FIG. 16. The Streptococcus thermophils which is not a host for T7 bacteriophage had very low measurements (below the M17 media) which indicates that Streptococcus thermophilus were not captured by the T7 beads.

### Example 12

### Detection of Antibiotic Resistant Bacteria and Capturing Them Using Bacteriophages Immobilized on a Solid Support

Biotinylated T7 bacteriophages were immobilized onto streptavidin magnetic beads. The T7-beads were stained with PMA and then incubated with E. coli and antibiotic resistant E. coli (ER2508). The beads were then washed to remove excess and unbound bacteria. Fresh LB media with and without the antibiotic tetracycline (10 ug/mL) were added followed by a 4.5-hour period for bacterial outgrowth. Using Resazurin, the results show that the T7-beads were able to capture both E. coli and antibiotic resistant E. coli (ER2508). In addition, this assay shows that antibiotic resistant E. coli (ER2508) can be captured and selected over non-antibiotic resistant strains from a broth containing antibiotics, providing a novel approach to testing of drug-resistance bacteria.

1. A method of detecting the presence of one or more types of cell in a sample comprising:
a) providing one or more types of viruses, wherein each of said one or more types of viruses bind to one or more specific types of receptor of said one or more types of cell if present in said sample to form complexes of virus and cell;
b) contacting said sample suspected of comprising said cell with said one or more types of viruses, wherein said one or more types of virus form complexes with said one or more types of cell if present in said sample;
c) contacting said one or more types of viruses with a first set of one or more compounds to generate one or more types of viruses comprising a nucleic acid comprising said first compound;
d) contacting said one or more types of cell with a second set of one or more compounds, wherein said second set of one or more compounds preferentially interacts with one or more components of said one or more types of cell in the presence of said one or more types of viruses; e) detecting said complexes by detecting said second set of one or more compounds or modifications of said second set of one or more compounds by said one or more types of cell, wherein said complexes are indicative of the presence of said one or more types of cell in said sample.

2. The method of claim 1, wherein upon contacting said one or more types of viruses with said first set of one or more compounds inhibits replication of one or more types of viruses in said one or more types of cell.

3. The method of claim 1, wherein contacting said one or more types of viruses with a first set of one or more compounds is done prior to contacting said sample suspected of comprising said cell with said one or more types of viruses.

4. The method of claim 1, wherein said one or more types of cell are one or more types of bacteria, and wherein said one or more types of viruses are one or more types of bacteriophage that bind to one or more specific types of receptor of said one or more types of bacteria.

5. The method of claim 4, wherein second set of one or more compounds preferentially binds to the nucleic acid of said one or more types of bacteria.

6. The method of claim 5, wherein second set of one or more compounds preferentially binds to the nucleic acid of said one or more types of bacteria after the formation of said complexes of bacteriophage and bacteria.

7. The method of claim 1, wherein said first set of one or more compounds comprises a first detectable label.

8. The method of claim 1, wherein said detecting further comprises detecting said first set of one or more compounds.

9. The method of claim 1, wherein said first set of one or more compounds is selected from the group consisting of propidium iodide, ethidium bromide, propidium monoazide, ethidium monoazide, a combination of, and derivatives thereof.

10. The method of claim 1, wherein said second set of one or more compounds interact with cellular nucleic acid.

11. The method of claim 1, wherein said second set of one or more compounds comprise a second detectable label.

12. The method of claim 1, wherein said second set of one or more compounds comprise a substrate for cellular enzymes.

13. The method of claim 1, wherein said second set of one or more compounds is a redox compound.

14. The method of claim 1, wherein said second set of one or more compounds is Resazurin, Resorufin, Dihydroresorufin, or a combination thereof.

15. The method of claims 14, wherein said modification of said second set of one or more compounds comprise a change in the oxidative state of Resazurin, Resorufin, Dihydroresorufin, or a combination thereof

16. The method of claim 15, wherein said detecting modifications of said second set of one or more compounds comprise measuring the change of the oxidative status of Resazurin, Resorufin, Dihydroresorufin or a combination thereof

17. The method of claim 1, further comprising measuring any modifications of said second set of one or more compounds by said one or more types of cell over a period of time and determining the kinetic profile of said modification of said second set of one or more compounds, wherein the kinetic profile is indicative of the presence of said one or more types of cell.

18. The method of claim 4, wherein said method is carried out at a temperature between about 20° C. and about 50° C.

19. (canceled)

20. The method of claim 1, wherein said second set of one or more compounds comprise Calcein derivative.

21. The method of claim 1, wherein said second set of one or more compounds comprises an antibody specific for a cellular protein.

22. (canceled)

23. The method of claim 4, further comprising incubating said complexes of said one or more types of bacteria and bacteriophage in a media comprising one or more bactericide or bacteriostatic agent prior to detecting said complexes.

24. (canceled)

25. The method of claim 23, wherein said one or more types of bacteriophages are immobilized on a solid support, and wherein complexes of said one or more types of bacteria and bacteriophage are immobilized on a solid support upon binding of the one or more types of bacteriophages to one or more specific types of receptor of said one or more types of bacteria.

26. A method of detecting one or more bactericide or bacteriostatic agent resistant bacteria in a sample comprising:
a) providing a first sample comprising one or more types of bacteriophages, wherein each of said one or more types of bacteriophages bind to one or more specific types of receptor of one or more types of said bacteria if present in said sample to form complexes of bacteria and bacteriophage; and wherein said one or more types of bacteriophages are incapable of replicating inside said one or more types of bacteria;
b) providing a second sample suspected of comprising one or more types of said bacteria, wherein said one or more types of said bacteria are susceptible to infection by said one or more types of bacteriophage;
c) contacting said first sample with said second sample to form a sample mixture, wherein said one or more types of bacteriophages if present in said first sample forms one or more complexes with said one or more types of bacteria in said sample mixture;
d) incubating said sample mixture in a media comprising one or more bactericide or bacteriostatic agent;
e) contacting one or more compounds to said media;
f) measuring any change of said one or more compounds in said media;
wherein any change of said one or more compounds is indicative of the presence or absence of one or more types of bacteriophage infected bacteria in said sample mixture, and wherein the presence or absence of one or more types of bacteriophage infected bacteria in said sample mixture is indicative of the presence or absence of one or more types of bactericide or bacteriostatic agent resistant bacteria in said second sample.

27.-30. (canceled)

31. The method of claim 26, further comprising measuring any change of said one or more compounds in presence of said sample mixture over a period of time and determining the kinetic profile of said change of said one or more compounds, wherein the kinetic profile is indicative of the presence or absence of one or more types of bacteriophage infected bacteria in said sample mixture, and wherein the presence or absence of one or more types of bacteriophage infected bacteria in said sample mixture is indicative of the presence or absence of one or more types of bacteria in said second sample.

32. The method of claim 26, further comprising comparing said kinetic profile of the change said one or more compounds with a kinetic profile of the change said one or more compounds in the presence of one or more types of bacteria susceptible to said one or more bactericide or bacteriostatic agent, wherein a difference between kinetic profiles is indicative of the presence of one or more types of bacteria resistant to one or more bactericide or bacteriostatic agent in said second sample.

33. The method of claim 26, wherein said one or more compounds is an enzymatic substrate of said one or more types of bacteria.

34. The method of claim 26, wherein said one or more compounds is a redox compound.

35.-38. (canceled)

39. The method of claim 26, wherein said one or more types of bacteriophage is chemically treated or genetically modified.

40.-48. (canceled)

49. A kit for detecting the presence of one or more types of cell in a sample using the method of claim 26, comprising:
a) a first reagent comprising one or more types of viruses, wherein each of said one or more types of viruses bind to one or more specific types of receptor of said one or more types of cell if present in said sample to form complexes of viruses and cell, wherein said one or more types of viruses are incapable of replicating inside said one or more types of cell;
b) a second reagent comprising one or more compounds preferentially interacts with one or more components of said cell in the presence of said one or more types of viruses of step (a);
c) buffers;
d) instruction for detecting said complexes by detecting said one or more compounds or modifications of said one or more compounds by said cell, wherein detecting said complexes is indicative of the presence of said one or more types of cell in said sample.
